(19) United States
(12) Patent Application Publication
(10) Pub. No.: US 2026/0271925 A1
(43) Pub. Date: Sep. 17, 2026

(54) DYSREGULATION OF PLANKTONIC BACTERIAL METABOLISM

(71) Applicant: Lixa Limited, Nedlands, Western Australia (AU)

(72) Inventors: Andrew Barker, Melville (AU); Maud Eijkenboom, Melville (AU); Clark Haese, Joondanna (AU); Kathryn Green, Melville (AU); Catherine Abberton, Perth (AU)

(21) Appl. No.: 19/470,864

(22) PCT Filed: May 9, 2024
(86) PCT No.: PCT/AU2024/050455

§ 371 Date: Sep. 30, 2025

(30) Foreign Application Priority Data
May 9, 2023 (AU) 2023901398

(51) Int. Cl. A01N 37/36; A01P 1/00; A61K 45/06

(52) U.S. Cl. CPC A01N 37/36; A01P 1/00; A61K 45/06

(57) ABSTRACT

A composition comprising a 2-hydroxycarboxylic acid for dysregulating the metabolism of planktonic bacteria, and uses of the same.

### TECHNICAL FIELD

The present disclosure relates to compositions comprising a preferred enantiomer of a 2-hydroxycarboxylic acid for the dysregulation of planktonic bacterial metabolism and the sensitising of planktonic bacteria to antimicrobial compounds, and methods of dysregulating planktonic bacterial metabolism and sensitising planktonic bacteria to antimicrobial compounds using the aforementioned compositions.

### BACKGROUND ART

In nature, bacteria may alternate between two main modes of growth: a unicellular life phase, in which the cells are free-swimming (planktonic), and a multicellular life phase, in which the cells are sessile and live in a colony, such as a biofilm. While a unicellular life phase allows for bacterial dispersion and the colonization of new environments, biofilms allow sessile cells to live in a coordinated, more permanent manner that favours survival and ongoing proliferation. In this alternating cycle, bacteria accomplish two physiological transitions via differential gene expression: (i) from planktonic cells to sessile cells within a biofilm, and (ii) from sessile to detached, newly planktonic cells.

Sessile bacteria differ in their metabolism from planktonic bacteria due to physiochemical conditions and physiological characteristics expressed to aid their survival within in a constrained environment (such as the biofilm). Estimates vary, but some suggest that as much as 40% of the genes of a bacterium may undergo up or down regulation in the transition from the planktonic to the biofilm state and vice versa. In addition, bacteria can develop and deploy resistance mechanisms to treatments, such as antibiotics and biocides, which undermine therapies and decontamination activities. The resistance mechanisms deployed by sessile biofilm bacteria can differ from the resistance mechanisms deployed by planktonic bacteria.

The presence of planktonic bacteria can be detrimental in healthcare, drinking water distribution systems, food, and marine industries, etc. For example, in food industries pathogenic bacteria may be present inside of processing facilities, leading to food spoilage and endangering consumer's health. In hospital settings, resistant and non-resistant planktonic bacteria have also been shown to persist on medical device surfaces, in patient's tissues or as airborne or fluid borne unicellular bacteria causing persistent infections. In addition, planktonic bacteria contribute to life-threatening infections and diseases in humans or animals such as sepsis, water-borne or food-borne diarrhoeal disease, urinary tract infections, lung infections, otitis media, periodontitis, sinusitis, chronic wounds, ocular infections, sexually transmitted diseases, bacterial meningitis, and osteomyelitis, as well as vaginosis. When these bacteria are resistant to bactericidal agents, major healthcare and contamination problems arise.

There is a need for clearing of recalcitrant bacterial infection present across all of nature including human disease, crop maintenance, commercial animal farms, animal disease and biofouling, including marine biofouling, biofouling of membranes, sensors and pipework, wind turbines etc.

Antimicrobial resistance may cause a decline in the efficacy of applied therapeutics, which may be an antibiotic, or agent with a different mode of antimicrobial action, such as a bacteriophage, antiseptic, saline, chlorine-based compound (such as bleach), an iodine-based compound, a copper-based compound, heavy metal etc. used to aid clearance of the planktonic bacteria.

Bacterial biofilms are complex communities of bacteria that are attached to a surface and/or to each other held together by self-produced polymer matrices mainly composed of polysaccharides, secreted proteins, and extracellular DNAs. The formation of a biofilm can begin with the attachment of free-floating planktonic microorganisms to a surface. Once colonization has begun, the biofilm grows through a combination of cell division and recruitment. If planktonic bacteria can be removed or killed before a biofilm develops, biofilm formation and other forms of bacterial persistence may be avoided. Furthermore, as biofilms can liberate individual bacterial cells to colonize other spaces where new biofilms can be formed, suppression, disruption or killing of the liberated bacteria before they form new biofilms would be advantageous.

There is a need for alternative treatments to assist in the disruption, suppression and/or killing of planktonic bacteria; or at least the provision of sensitising agents to complement additional bacterial treatments, which may prevent the development of, or disrupt, the resistance mechanisms deployed by bacteria. The present disclosure seeks to provide an improved or alternative method for disruption, suppression and killing of planktonic bacteria, particularly methods that may be used in conjunction with antimicrobial compounds, such as antibiotics or biocides.

The previous discussion of the background art is intended to facilitate an understanding of the present disclosure only. The discussion is not an acknowledgement or admission that any of the material referred to is or was part of the common general knowledge as at the priority date of the application.

### SUMMARY OF INVENTION

The present disclosure provides a composition for the dysregulation of planktonic bacterial metabolism, said composition comprising a preferred enantiomer of a 2-hydroxycarboxylic acid.

The present disclosure further provides a composition for the dysregulation of planktonic bacterial metabolism, said composition comprising a preferred enantiomer of a 2-hydroxycarboxylic acid and an antimicrobial compound.

The dysregulation of planktonic bacterial metabolism as measured by, amongst other examples, a reduction in companion antimicrobial MIC, enhanced in vivo antimicrobial efficacy, or in the aerobic respiration of metabolically active cells (the reduction of non-fluorescent resazurin to the highly fluorescent resorufin) may lead to sensitisation of the planktonic bacteria to antimicrobial compounds, and/or resistance breaking of planktonic bacteria to antimicrobial compounds. The dysregulation of planktonic bacterial metabolism may also lead to enhanced bacterial killing or enhanced infection mitigation when an antimicrobial compound is introduced to the planktonic bacteria, either while the preferred enantiomer of a 2-hydroxycarboxylic acid is dysregulating the metabolism of the planktonic bacteria (preferably leading to sensitisation), or after the preferred enantiomer of a 2-hydroxycarboxylic acid has dysregulated the metabolism of the planktonic bacteria (preferably leading to sensitisation). The dysregulation of planktonic bacterial metabolism may be measured as metabolic suppression, metabolic enhancement, or metabolic suppression followed by metabolic enhancement.

The present disclosure further provides a composition for the dysregulation of planktonic bacterial metabolism of planktonic bacteria that are not resistant to one or more antimicrobial compounds (i.e. planktonic bacteria that are sensitive to the antimicrobial compounds, termed “non-resistant planktonic bacteria”), said composition comprising a preferred enantiomer of a 2-hydroxycarboxylic acid, wherein the preferred enantiomer of a 2-hydroxycarboxylic acid induces dysregulation of the planktonic bacterial metabolism and sensitises the bacteria to an antimicrobial compound.

The present disclosure further provides a composition for the dysregulation of planktonic bacterial metabolism of planktonic bacteria that are resistant to one or more antimicrobial compounds (termed “resistant planktonic bacteria”), said composition comprising a preferred enantiomer of a 2-hydroxycarboxylic acid, wherein the preferred enantiomer of a 2-hydroxycarboxylic acid induces dysregulation of the planktonic bacterial metabolism and sensitises the bacteria to an antimicrobial compound.

The present disclosure further provides a composition for sensitising planktonic bacteria to an antimicrobial compound, said composition comprising a preferred enantiomer of a 2-hydroxycarboxylic acid, wherein the preferred enantiomer of a 2-hydroxycarboxylic acid induces dysregulation of the planktonic bacterial metabolism.

The present disclosure further provides a composition for sensitising planktonic bacteria to an antimicrobial compound, said composition comprising a preferred enantiomer of a 2-hydroxycarboxylic acid and the antimicrobial compound, wherein the preferred enantiomer of a 2-hydroxycarboxylic acid induces dysregulation of the planktonic bacterial metabolism.

The disclosure further provides a composition for enhancing the efficacy of an antimicrobial compound to decontaminate a surface comprising planktonic bacteria, said composition comprising a preferred enantiomer of a 2-hydroxycarboxylic acid, wherein the preferred enantiomer of a 2-hydroxycarboxylic acid induces dysregulation of the planktonic bacterial metabolism.

The disclosure further provides a composition for enhancing the efficacy of an antimicrobial compound to decontaminate a surface comprising planktonic bacteria, said composition comprising a preferred enantiomer of a 2-hydroxycarboxylic acid and the antimicrobial compound, wherein the preferred enantiomer of a 2-hydroxycarboxylic acid induces dysregulation of the planktonic bacterial metabolism.

The antimicrobial compound in the compositions of the present invention may be added with the preferred enantiomer of a 2-hydroxycarboxylic acid or added after the preferred enantiomer of a 2-hydroxycarboxylic acid has been introduced to the planktonic bacteria. Optionally, if the antimicrobial compound is added after the preferred enantiomer of a 2-hydroxycarboxylic acid, the planktonic bacteria have been sensitised to the antimicrobial compound by the preferred enantiomer of a 2-hydroxycarboxylic acid.

In the compositions of the present invention, optionally the dysregulation of planktonic bacterial metabolism results in the sensitisation of the bacteria to an antimicrobial compound. In the compositions of the present invention, optionally the dysregulation of the planktonic bacterial metabolism is suppression of the planktonic bacterial metabolism, or suppression of bacterial metabolism followed by enhancement of bacterial metabolism.

The planktonic bacteria may be resistant planktonic bacteria, non-resistant planktonic bacteria, or a combination of resistant and non-resistant planktonic bacteria.

The compositions of the present disclosure may comprise from 0.001% to 100% of total 2-hydroxycarboxylic acid. The rest of the composition may comprise carriers, diluents or excipients, and/or other active agents. In one aspect, the % of the unpreferred enantiomer of a 2-hydroxycarboxylic acid in the composition is less than 20%.

The antimicrobial compound may be a bactericidal, bacteriostatic, antibiofilm or antiseptic antimicrobial compound. The antimicrobial compound may be an antibiotic, or agent with a different mode of antimicrobial action, such as a bacteriophage, antiseptic, saline, chlorine-based compound (such as bleach), an iodine-based compound, a copper-based compound, heavy metal etc.

The compositions of the present disclosure comprising a preferred enantiomer of a 2-hydroxycarboxylic acid may be used to dysregulate (for example by suppressing) the metabolism of planktonic bacteria, and optionally sensitise the planktonic bacterium to an antimicrobial compound, on both living and non-living surfaces, as well as free floating planktonic bacteria in fluids and gases (including air).

The present disclosure further provides a method for the dysregulation of planktonic bacterial metabolism comprising the step of:
i. administering a preferred enantiomer of a 2-hydroxycarboxylic acid to the planktonic bacterium.

The disclosure further provides a method for the dysregulation of planktonic bacterial metabolism comprising the step of:
i. administering a preferred enantiomer of a 2-hydroxycarboxylic acid to the planktonic bacterium
   wherein the preferred enantiomer of a 2-hydroxycarboxylic acid is in combination with an antimicrobial compound.

The disclosure further provides a method for sensitising planktonic bacteria to an antimicrobial compound, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism.

The disclosure further provides a method for sensitising planktonic bacteria to an antimicrobial compound, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

The disclosure further provides a method for enhancing the efficacy of an antimicrobial compound to decontaminate a surface comprising planktonic bacteria, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism.

The disclosure further provides a method for enhancing the efficacy of an antimicrobial compound to decontaminate a surface comprising planktonic bacteria, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

The present disclosure further provides a method to treat or prevent a bacterial infection by planktonic bacteria, comprising the step of:
i. administering a preferred enantiomer of a 2-hydroxycarboxylic acid to the site of infection
   wherein the preferred enantiomer of a 2-hydroxycarboxylic acid induces dysregulation of the planktonic bacterial metabolism.

The disclosure further provides a method to treat or prevent a bacterial infection by planktonic bacteria, comprising the step of:
i. administering a preferred enantiomer of a 2-hydroxycarboxylic acid to the site of infection
   wherein the preferred enantiomer of a 2-hydroxycarboxylic acid is in combination with an antimicrobial compound, and wherein the preferred enantiomer of a 2-hydroxycarboxylic acid induces dysregulation of the planktonic bacterial metabolism.

The present disclosure further provides a composition comprising a preferred enantiomer of a 2-hydroxycarboxylic acid for use in treating or preventing a bacterial infection in a subject, wherein the bacterial infection is by planktonic bacteria, and wherein the preferred enantiomer of a 2-hydroxycarboxylic acid induces dysregulation of the planktonic bacterial metabolism.

The present disclosure further provides a composition comprising a preferred enantiomer of a 2-hydroxycarboxylic acid for use in treating or preventing an infection in a subject, in combination with an antimicrobial compound, wherein the infection is by planktonic bacteria, and wherein the preferred enantiomer of a 2-hydroxycarboxylic acid induces dysregulation of the planktonic bacterial metabolism.

The present disclosure further provides a composition comprising a preferred enantiomer of a 2-hydroxycarboxylic acid for use in treating or preventing a bacterial infection in a subject, wherein the bacterial infection is by planktonic bacteria, wherein the composition sensitises the planktonic bacterium to an antimicrobial compound, and wherein the preferred enantiomer of a 2-hydroxycarboxylic acid induces dysregulation of the planktonic bacterial metabolism.

The present disclosure further provides a composition comprising a preferred enantiomer of a 2-hydroxycarboxylic acid for use in treating or preventing an infection in a subject, in combination with an antimicrobial compound, wherein the infection is by planktonic bacteria, wherein the composition sensitises the planktonic bacterium to the antimicrobial compound, and wherein the preferred enantiomer of a 2-hydroxycarboxylic acid induces dysregulation of the planktonic bacterial metabolism.

The present disclosure provides for the use of a preferred enantiomer of a 2-hydroxycarboxylic acid in the manufacture of a composition for the dysregulation of planktonic bacterial metabolism.

The present disclosure provides for the use of a preferred enantiomer of a 2-hydroxycarboxylic acid in the manufacture of a composition for the dysregulation of planktonic bacterial metabolism, in combination with an antimicrobial compound.

The present disclosure further provides for the use of a preferred enantiomer of a 2-hydroxycarboxylic acid for the dysregulation of planktonic bacterial metabolism.

The present disclosure further provides for the use of a preferred enantiomer of a 2-hydroxycarboxylic acid for the dysregulation of planktonic bacterial metabolism, in combination with an antimicrobial compound.

The present disclosure further provides for the use of a preferred enantiomer of a 2-hydroxycarboxylic acid for sensitising planktonic bacteria to an antimicrobial compound, wherein the preferred enantiomer of a 2-hydroxycarboxylic acid induces dysregulation of the planktonic bacterial metabolism.

The present disclosure further provides for the use of a preferred enantiomer of a 2-hydroxycarboxylic acid for sensitising planktonic bacteria to an antimicrobial compound, wherein the preferred enantiomer of a 2-hydroxycarboxylic acid is in combination with the antimicrobial compound, and wherein the preferred enantiomer of a 2-hydroxycarboxylic acid induces dysregulation of the planktonic bacterial metabolism.

The present disclosure provides for the use of a preferred enantiomer of a 2-hydroxycarboxylic acid in the manufacture of a composition for sensitising planktonic bacteria to an antimicrobial compound, wherein the preferred enantiomer of a 2-hydroxycarboxylic acid induces dysregulation of the planktonic bacterial metabolism.

The present disclosure provides for the use of a preferred enantiomer of a 2-hydroxycarboxylic acid in the manufacture of a composition for sensitising planktonic bacteria to an antimicrobial compound, wherein the preferred enantiomer of a 2-hydroxycarboxylic acid is in combination with the antimicrobial compound, and wherein the preferred enantiomer of a 2-hydroxycarboxylic acid induces dysregulation of the planktonic bacterial metabolism.

In the uses above, optionally the dysregulation of the planktonic bacterial metabolism is suppression of the planktonic bacterial metabolism.

The preferred enantiomer of a 2-hydroxycarboxylic acid may be in the same composition as the antimicrobial compound or may be in separate compositions. The antimicrobial compound may be a bactericidal, bacteriostatic, antibiofilm or antiseptic antimicrobial compound. The antimicrobial compound may be a bactericidal or bacteriostatic antibiotic, or agent with a different mode of antimicrobial action, such as a bacteriophage, antibiofilm, quorum-sensing inhibitor, antiseptic, saline, chlorine-based compound (such as bleach), an iodine-based compound, a copper-based compound, or heavy metal.

The present disclosure provides a kit for the suppression of planktonic bacterial metabolism comprising:
a) a preferred enantiomer of a 2-hydroxycarboxylic acid; and
b) instructions for use.

The present disclosure provides a kit for the suppression of planktonic bacterial metabolism comprising:
a) a preferred enantiomer of a 2-hydroxycarboxylic acid; and
b) instructions for use
   wherein the preferred enantiomer of a 2-hydroxycarboxylic acid is in combination with an antimicrobial compound.

The present disclosure provides a kit for sensitising planktonic bacteria to an antimicrobial compound, comprising:
a) a preferred enantiomer of a 2-hydroxycarboxylic acid; and
b) instructions for use.
   wherein the preferred enantiomer of a 2-hydroxycarboxylic acid induces dysregulation of the planktonic bacterial metabolism.

The present disclosure provides a kit for sensitising planktonic bacteria to an antimicrobial compound, comprising:
a) a preferred enantiomer of a 2-hydroxycarboxylic acid; and
b) instructions for use
   wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

In the methods, uses and kits of the present invention, the antimicrobial compounds may be added with the preferred enantiomer of a 2-hydroxycarboxylic acid, or added after the preferred enantiomer of a 2-hydroxycarboxylic acid has been introduced to the planktonic bacteria. Optionally, if the antimicrobial compound is added after the preferred enantiomer of a 2-hydroxycarboxylic acid, the resistant and/or non-resistant planktonic bacteria have been sensitised to the antimicrobial compound by the preferred enantiomer of a 2-hydroxycarboxylic acid.

In the methods, uses and kits of the present invention, optionally the dysregulation of planktonic bacterial metabolism results in the sensitisation of the bacteria to an antimicrobial compound. In the methods, uses and kits of the present invention, optionally the dysregulation of the planktonic bacterial metabolism is suppression of the planktonic bacterial metabolism, or suppression of bacterial metabolism followed by enhancement of bacterial metabolism.

The planktonic bacteria may be resistant planktonic bacteria, non-resistant planktonic bacteria, or a combination of resistant and non-resistant planktonic bacteria.

In the embodiments above, the preferred enantiomer of a 2-hydroxycarboxylic acid is D-lactic acid or a pharmaceutically acceptable salt thereof.

### BRIEF DESCRIPTION OF THE DRAWINGS

Further features of the present disclosure are more fully described in the following description of several non-limiting embodiments thereof. This description is included solely for the purposes of exemplifying the present disclosure. It should not be understood as a restriction on the broad summary, disclosure or description of the disclosure as set out above. The description will be made with reference to the accompanying drawings in which:

FIGS. 1A-B are graphs of the outcome of adding 2-fold decreasing concentrations of D-Lactic acid to planktonic cultures of P. aeruginosa clinical isolates of mixed phenotype. FIGS. 1A, 1C and 1E show the measurement of culture absorbance as a proxy for the density of suspended bacteria. As can be seen from each histogram, the treatment does not lead to a significant decrease in culture absorbance across the concentration range of D-lactic acid evaluated. FIGS. 1B, 1D and 1F however show that the same treatment range results in a breakpoint between 0.25 mg/mL and 0.5 mg/mL D-Lactic acid when metabolic rate (Resazurin reduction to the fluorescent Resorufin) is used as a readout. FIG. 1A shows absorbance (culture density) P. aeruginosa clinical isolates exhibiting Smooth colony morphology (n=8) after treatment with the indicated D-Lactic acid concentration. FIG. 1B shows the relative fluorescence measured after the addition of Resazurin to the same P. aeruginosa clinical isolates with Smooth colony morphology (n=8). A break point in metabolism for these cultures is apparent between 0.25 mg/mL and 0.5 mg/mL treatments with D-Lactic acid. FIG. 1C shows absorbance (culture density) of P. aeruginosa clinical isolates exhibiting Rough colony morphology (n=5) after treatment with the indicated D-Lactic acid concentration range. FIG. 1D shows the relative fluorescence measured after the addition of Resazurin to the same Rough colony morphology P. aeruginosa clinical isolates (n=5). A break point in metabolic response of these cultures is apparent between treatments of 0.25 mg/mL and 0.5 mg/mL of D-Lactic acid. FIG. 1E shows absorbance (culture density) of P. aeruginosa clinical isolates exhibiting Mucoid colony morphology (n=11) after treatment with the indicated D-Lactic acid concentration range. FIG. 1F shows the relative fluorescence measured after the addition of Resazurin to the same Mucoid colony morphology P. aeruginosa clinical isolates (n=11). A break point in metabolic response is apparent in treatments between 0.25 mg/mL and 0.5 mg/mL of D-Lactic acid.

FIG. 2 is a graph showing D-Lactic acid enhances metabolic activity suppression in the presence of ceftriaxone. A: S. aureus 29213 (MSSA). B: S. aureus 510 (MRSA). Relative metabolic activity is indicated in black for ceftriaxone-only treatments and in grey for ceftriaxone plus A: 10 mM D-Lactic acid, or B: 0.1 mM D-Lactic acid.

FIG. 3 is a graph showing D-Lactic acid enhances metabolic activity suppression in the presence of meropenem. A: S. aureus 29213 (MSSA). B: S. aureus 510 (MRSA). Relative metabolic activity is indicated in black for meropenem-only treatments and in grey for meropenem plus 10 mM D-Lactic acid.

FIG. 4 is a graph showing D-Lactic acid enhances metabolic activity suppression in the presence of methicillin. A: S. aureus 29213 (MSSA). B: S. aureus 510 (MRSA). Relative metabolic activity is indicated in black for methicillin-only treatments and in grey for methicillin plus 10 mM D-Lactic acid.

FIG. 5 is a graph showing D-Lactic acid enhances metabolic activity suppression in the presence of rifampicin. A: S. aureus MSSA293 (MSSA). B: S. aureus 510 (MRSA). Relative metabolic activity is indicated in black for rifampicin-only treatments and in grey for rifampicin plus 0.16 mM D-Lactic acid.

FIG. 6 is a graph showing D-Lactic acid enhances metabolic activity suppression in the presence tetracycline in S. aureus 510 (MRSA). Relative metabolic activity is indicated in black for rifampicin-only treatment and in grey for tetracycline plus 10 mM D-Lactic acid.

FIG. 7 is a graph showing D-Lactic acid enhances metabolic activity suppression in the presence of vancomycin. A: S. aureus 29213 (MSSA). B: S. aureus 510 (MRSA). Relative metabolic activity is indicated in black for vancomycin-only treatments and in grey for vancomycin plus A: 10 mM D-Lactic acid, or B: 1.0 mM D-Lactic acid.

FIG. 8 is a graph showing D-Lactic acid enhances metabolic activity suppression in the presence of antibiotics in K. pneumoniae WACC 790. A: Ciprofloxacin. B: Colistin. Relative metabolic activity is indicated in black for antibiotic-only treatments and in grey for antibiotic plus A: 0.1 mM D-Lactic acid, or B: 10 mM D-Lactic acid.

FIG. 9 is a graph showing D-Lactic acid enhances metabolic activity suppression in the presence of antibiotics in S. pneumoniae D39. A: Amoxicillin. B: Ampicillin. Relative metabolic activity is indicated in black for antibiotic-only treatments and in grey for antibiotic plus 10 mM D-Lactic acid.

FIG. 10 is a graph showing D-Lactic acid enhances metabolic activity suppression in the presence of antibiotics in S. pneumoniae D39. A: Ceftriaxone. B: Tetracycline. C: Vancomycin. Relative metabolic activity is indicated in black for antibiotic-only treatments and in grey for antibiotic plus 10 mM D-Lactic acid.

FIG. 11 is a histogram of the outcome of adding 2-fold decreasing concentrations of D-Lactic acid or L-Lactic acid to planktonic cultures of P. aeruginosa WACC91.

FIG. 12 is a graph of the total bacterial counts from blood of control and treated mice at 4 hours post-infection. Significant differences between control and treated groups were obtained by unpaired t-tests (two-tailed; *=p<0.05; **=p<0.01; ns=not significant). Dashed horizontal line denotes limit of detection.

FIG. 13 provides representative images of 2 CD1 male mice (ventral and dorsal) at indicated times post-IP challenge with col^{R} E. coli Xen14, demonstrating that treatment with Sodium D-Lactate and colistin was more effective than treatment with colistin alone.

FIG. 14 is a graph of a survival analysis of infected mice. Five out of eight mice in the Sodium D-Lactate+1 mg/kg colistin combination group and three out eight mice from the 1 mg/kg colistin treatment group survived the infection by col^{R} E. coli Xen14 up to the end of the experiment (72 h post-infection). However, all control mice, and those treated with Sodium D-Lactate alone, succumbed to infection by 12 h post-challenge.

### DESCRIPTION OF INVENTION

### Detailed Description of the Invention

2-hydroxycarboxylic acids, such as lactic acid, are known to be bactericidal due to their ability to reduce the environmental pH and disrupt the integrity of the cell membrane. These antibacterial properties have been used in food production.

However, the present disclosure has surprisingly found that one enantiomer of enantiomeric 2-hydroxycarboxylic acids, referred to in the present application as the “preferred enantiomer of a 2-hydroxycarboxylic acid”, “preferred enantiomer” or the “preferred 2-hydroxycarboxylic acid”, is effective in the dysregulation of planktonic bacterial metabolism. This metabolic dysregulation effect is not provided by the other enantiomer of the enantiomeric 2-hydroxycarboxylic acid, also known as the “unpreferred enantiomer of a 2-hydroxycarboxylic acid”, “unpreferred enantiomer” or the “unpreferred 2-hydroxycarboxylic acid”. It has been found that in some situations the unpreferred enantiomer is not only not active in metabolic dysregulation, but can potentially prevent the preferred enantiomer from having its effect. Preferably the dysregulation of the planktonic bacterial metabolism is suppression of the planktonic bacterial metabolism.

The metabolic dysregulation effect is not a bactericidal effect; both enantiomers of enantiomeric 2-hydroxycarboxylic acids such as lactic acid are known to have equal bactericidal effects. In some cases, the bactericidal effects of the two enantiomers are due to their effects on pH. In the present disclosure, the preferred enantiomer is used at a concentration lower than would be used if the preferred enantiomer was being used for its bactericidal effects.

The term “dysregulation of metabolism” includes both suppression of the vitality of the bacteria and enhancement of the vitality of the bacteria. The dysregulation may be a change in aerobic respiration, a change in metabolic activity, suppression of NADH or NADPH generation; a change in the viability of the bacteria; a change in the development of resistance to antimicrobial compounds (bacteriostatic and bactericidal compounds); and/or a change in the sensitivity of the bacteria to antimicrobial compounds (bacteriostatic and bactericidal compounds).

It has been found that the addition of the preferred enantiomer of a 2-hydroxycarboxylic acid in the absence of an antimicrobial compound generally induces the suppression of planktonic bacterial metabolism; however, in some cases it leads to enhancement of metabolism. This bacterial metabolism dysregulation has been found to lead to the planktonic bacteria becoming more sensitive to antimicrobial compounds. Optionally, the dysregulation of planktonic bacterial metabolism results in the sensitisation of the bacteria to an antimicrobial compound.

Once the planktonic bacteria are in the presence of both the preferred enantiomer of a 2-hydroxycarboxylic acid and an antimicrobial compound, in some cases the metabolism of the planktonic bacteria continues to be suppressed. In other cases, in the presence of both the preferred enantiomer of a 2-hydroxycarboxylic acid and an antimicrobial compound, the metabolism of the planktonic bacteria may be enhanced.

The dysregulation of metabolism of the planktonic bacteria by the preferred enantiomer of a 2-hydroxycarboxylic acid may be one of the following:
suppression of metabolism in the presence of the 2-hydroxycarboxylic acid alone, and suppression of metabolism in the presence of the 2-hydroxycarboxylic acid and an antimicrobial compound;
suppression of metabolism in the presence of the 2-hydroxycarboxylic acid alone, but enhancement of metabolism in the presence of the 2-hydroxycarboxylic acid and an antimicrobial compound;
enhancement of metabolism in the presence of the 2-hydroxycarboxylic acid alone, and enhancement of metabolism in the presence of the 2-hydroxycarboxylic acid and an antimicrobial compound;
enhancement of metabolism in the presence of the 2-hydroxycarboxylic acid alone, but suppression of metabolism in the presence of the 2-hydroxycarboxylic acid and an antimicrobial compound;
no effect on metabolism in the presence of the 2-hydroxycarboxylic acid alone, but suppression of metabolism in the presence of the 2-hydroxycarboxylic acid and an antimicrobial compound;
no effect on metabolism in the presence of the 2-hydroxycarboxylic acid alone, but enhancement of metabolism in the presence of the 2-hydroxycarboxylic acid and an antimicrobial compound.

The timing of the exposure of the bacteria to the 2-hydroxycarboxylic acid may be: before introduction of the planktonic bacteria to the antimicrobial compound, at the same time as the antimicrobial compound, or after introduction of the planktonic bacteria to the antimicrobial compound.

The term “suppression of planktonic bacterial metabolism” includes: suppression of the vitality of the bacteria, for example suppression of aerobic respiration, suppression of mitochondrial metabolic activity, suppression of NADH or NADPH generation; suppression of the viability of the bacteria; suppression of the development of resistance to antimicrobial compounds (bacteriostatic and bactericidal compounds); and/or enhancing the sensitivity of the bacteria to antimicrobial compounds (bacteriostatic and bactericidal compounds). The suppression may be measured by comparing the metabolism of the bacteria in the presence of the preferred enantiomer of a 2-hydroxycarboxylic acid to the metabolism of the same bacteria in the absence of the preferred enantiomer of a 2-hydroxycarboxylic acid. If the bacterial metabolism is lower in the presence of the 2-hydroxycarboxylic acid, then suppression of planktonic bacterial metabolism has occurred. The % change is dependent on the metabolic parameter used (for example, resazurin reduction, or companion antimicrobial MIC reduction, or enhanced in vivo antimicrobial efficacy) and may be 1%, 5%, 10%, 15%, 20%, 25%, 30%, 35%, 40%, 45%, 50%, 55%, 60%, 65%, 70%, 75%, 80%, 85%, 90%, 95%, 97%, 98%, 99% or 100%, or any range of these.

The term “enhancement of planktonic bacterial metabolism” includes: enhancement of the vitality of the bacteria, for example enhancement of aerobic respiration, enhancement of mitochondrial metabolic activity, enhancement of NADH or NADPH generation; enhancement of the viability of the bacteria; and/or enhancing the sensitivity of the bacteria to antimicrobial compounds (bacteriostatic and bactericidal compounds). The enhancement may be measured by comparing the metabolism of the bacteria in the presence of the preferred enantiomer of a 2-hydroxycarboxylic acid to the metabolism of the same bacteria in the absence of the preferred enantiomer of a 2-hydroxycarboxylic acid. If the bacterial metabolism is higher in the presence of the 2-hydroxycarboxylic acid, then enhancement of planktonic bacterial metabolism has occurred. The % change is dependent on the metabolic parameter used (for example, resazurin reduction, or companion antimicrobial MIC reduction, or enhanced in vivo antimicrobial efficacy) and may be 1%, 5%, 10%, 15%, 20%, 25%, 30%, 35%, 40%, 45%, 50%, 55%, 60%, 65%, 70%, 75%, 80%, 85%, 90%, 95%, 97%, 98%, 99% or 100%, or any range of these. The enhancement of planktonic bacterial metabolism preferably occurs after metabolic suppression has occurred, preferably after addition of the antimicrobial compound. However, in some cases the enhancement of planktonic bacterial metabolism may occur on exposure to the preferred enantiomer of a 2-hydroxycarboxylic acid alone, without being prefaced by suppression and/or the presence of an antimicrobial compound.

The present disclosure has found that the preferred enantiomer of a 2-hydroxycarboxylic acid, including D-lactic acid, is a small water-soluble compound that can be used for the purpose of suppressing the metabolism of planktonic bacteria. Planktonic bacterial infections and contaminations may be treated by the preferred enantiomer of a 2-hydroxycarboxylic acid alone, through its ability to disrupt the metabolism of bacteria. In such cases, the metabolic disruption is preferably metabolic suppression.

Alternatively, the preferred enantiomer of a 2-hydroxycarboxylic acid can be used in combination with other antimicrobial compounds or therapies (both bactericidal and bacteriostatic antimicrobial compounds). The antimicrobial compound may be an antibiotic or non-antibiotic antimicrobial compound. The metabolic dysregulation activity of the preferred enantiomer of a 2-hydroxycarboxylic acid enhances the bactericidal, bacteriostatic, antibiofilm, or antiseptic efficacy of the antimicrobial compounds or therapies. In such cases, the metabolic disruption may be metabolic suppression, or metabolic enhancement. For example, the preferred enantiomer of a 2-hydroxycarboxylic acid may cause metabolic suppression, which is followed by metabolic enhancement on introduction of the antimicrobial compound. Alternatively, the preferred enantiomer of a 2-hydroxycarboxylic acid may cause metabolic suppression, which is maintained in the presence of the antimicrobial compound. However, in some cases the enhancement of planktonic bacterial metabolism may occur on exposure to the preferred enantiomer of a 2-hydroxycarboxylic acid alone, without being prefaced by suppression and/or the presence of an antimicrobial compound. In such cases, the metabolic enhancement may be followed by metabolic suppression in the presence of the 2-hydroxycarboxylic acid and an antimicrobial compound.

In the present disclosure, the preferred enantiomer of a 2-hydroxycarboxylic acid may act as an “antimicrobial resistance breaker” (ARB). ARBs are compounds that are administered along with an antibiotic to overcome the bacterial resistance to that antibiotic, either by improving the antibacterial drug efficacy or by inhibiting the bacterial resistance mechanism. If the preferred enantiomer of a 2-hydroxycarboxylic acid is acting as an ARB, the preferred enantiomer of a 2-hydroxycarboxylic acid may improve the efficacy of the antibiotic by reducing the minimum inhibitory concentration (MIC) of the antibiotic; for example, the preferred enantiomer of a 2-hydroxycarboxylic acid may reduce the MIC of an antibiotic to lower than the MIC of monotherapy of the antibiotic.

Without being held to any theory, it is believed that the dysregulation of metabolism induced by the preferred enantiomer of a 2-hydroxycarboxylic acid may allow the 2-hydroxycarboxylic acid to act as an ARB and improving the sensitivity of the bacteria to an antibiotic, and/or increase the efficacy of an antibiotic.

If the dysregulation of metabolism induced by the preferred enantiomer of a 2-hydroxycarboxylic acid is the suppression of metabolism, the effect of the 2-hydroxycarboxylic acid acting as an ARB may be to increase the amount of the antibiotic being held within the bacterial cell by a reduction in efflux, the bacteria being unable to modify the antibiotic once the antibiotic is internalised, or via another mechanism.

Once the bacterium is in the presence of both the preferred enantiomer of a 2-hydroxycarboxylic acid and an antimicrobial compound, if the metabolism of the planktonic bacteria is enhanced, the effect of the 2-hydroxycarboxylic acid acting as an ARB may be to increase uptake of the antimicrobial compound, or increase in the generation of free radicals.

The term “planktonic bacterium” as used herein refers to free-swimming or free-floating bacteria that are not located within a biofilm. Although the bacteria are free-swimming or free-floating, the swimming or floating may be on a solid surface. Thus, the planktonic bacteria may be present on a surface. Nevertheless, its presence on the surface is not within or bound to a biofilm and the planktonic bacteria is not adhered to the surface. At the micrometre level, a planktonic habitat for prokaryotes can also encompass water films around soil particles, saliva in the mouth, fluids in the intestinal lumen, serum in blood vessels, urine in the bladder and urinary tract, aqueous compositions flowing through plant xylem and/or phloem, surface water on plant leaves, in soil substrate etc. Planktonic bacteria may settle on a surface and eventually develop into a biofilm by adhering and then extruding extracellular matrix; however, the bacteria targeted by the present disclosure are those that have not yet developed the characteristics of a biofilm, or individual bacteria that are released from a biofilm to become free floating bacteria that can settle elsewhere to restart the biofilm process.

The planktonic bacteria of the present disclosure may be a bacterium that is not capable of developing into a biofilm and lives its life in the planktonic form.

The planktonic bacteria of the present disclosure may be a bacterium that has been released from a biofilm bound colony in a spontaneous or induced manner, such as due to biofilm matrix disruption.

Compared to bacteria within a biofilm, planktonic bacteria often have upregulated DNA repair genes (such as the bacterial SOS response genes). It has further been reported that bacteria released from biofilms to become planktonic may retain some or all of the characteristics of the biofilm-associated “parent” population, and thus will have increased resistance to antibiotics. These populations of released planktonic bacteria may be difficult to remove from situations that require abiosis and are often a source of repeated infection.

### Composition

The present disclosure provides a composition for the dysregulation of planktonic bacterial metabolism, said composition comprising a preferred enantiomer of a 2-hydroxycarboxylic acid, such as D-lactic acid.

The present disclosure further provides a composition for the dysregulation of planktonic bacterial metabolism, said composition comprising a preferred enantiomer of a 2-hydroxycarboxylic acid and an antimicrobial compound.

The present disclosure further provides a composition for sensitising planktonic bacteria to an antimicrobial compound, said composition comprising a preferred enantiomer of a 2-hydroxycarboxylic acid, wherein the preferred enantiomer of a 2-hydroxycarboxylic acid induces dysregulation of the planktonic bacterial metabolism.

The present disclosure further provides a composition for sensitising planktonic bacteria to an antimicrobial compound, said composition comprising a preferred enantiomer of a 2-hydroxycarboxylic acid and an antimicrobial compound, wherein the preferred enantiomer of a 2-hydroxycarboxylic acid induces dysregulation of the planktonic bacterial metabolism.

The dysregulation of bacterial metabolism may lead to sensitisation. The sensitisation may be through potentiation or synergy. In one aspect, the dysregulation of the planktonic bacterial metabolism is suppression of the planktonic bacterial metabolism, in another aspect it is enhancement of the planktonic bacterial metabolism, in yet another aspect it is one of suppression or enhancement, followed by the other of suppression or enhancement.

### 2-Hydroxycarboxylic Acids

The 2-hydroxycarboxylic acids of the present invention have a stereochemistry, and one enantiomer of the stereochemistry is preferred. The 2-hydroxycarboxylic acid of the disclosure that has the preferred stereochemistry is herein referred to as the “preferred enantiomer of a 2-hydroxycarboxylic acid”, “preferred enantiomer” or “preferred 2-hydroxycarboxylic acid”. The 2-hydroxycarboxylic acids are optionally hydrophilic 2-hydroxycarboxylic acids. In one aspect, the preferred enantiomer of a 2-hydroxycarboxylic acid is D-lactic acid. The preferred 2-hydroxycarboxylic acids of the present invention may be a salt of the preferred 2-hydroxycarboxylic acid, optionally a pharmaceutically acceptable salt.

Where the preferred 2-hydroxycarboxylic acid is not D-lactic acid, the preferred 2-hydroxycarboxylic acids to be utilised in the present disclosure may be those in which the absolute stereochemistry of the chiral centre to which the hydroxy group of the 2-hydroxycarboxylic acid corresponds to the absolute stereochemistry of the corresponding chiral centre in D-lactic acid, in terms of the 3-dimensional orientation of the hydroxy group, as depicted below:

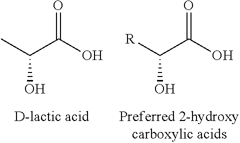

The preferred 2-hydroxycarboxylic acids of the above general formula may be those in which the substituent R is selected from the group consisting of: hydrogen, halogen (F, Cl, Br, I), methyl, ethyl, n-propyl, isopropyl, cyclopropyl, n-butyl, iso-butyl, sec-butyl, tert-butyl, cyclobutyl, n-pentyl, isopentyl, neopentyl, cyclopentyl, n-hexyl, 2-methylpentyl, 3-methylpentyl, 2,3-dimethylbutyl, 2,2-dimethylbutyl, cyclohexyl, n-heptyl, 2-methylhexyl, 3-methylhexyl, 2,3-dimethylpentyl, 2,4-dimethylpentyl, 2,2-dimethylpentyl, 3,3-dimethylpentyl, 3-ethylpentyl, 2,2,3-trimethylbutyl, cycloheptyl, phenyl, benzyl, furanyl, tetrahydrofuranyl, ethenyl, vinyl, allyl, crotyl, isopentenyl, 1-propenyl, 2-propenyl, 1-butenyl, 2-butenyl, 2-methylprop-2-enyl, hexenyl, butadienyl, pentadienyl, hexadienyl, 2,3-dimethyl-2-butenyl, heptenyl, octenyl, octatrienyl, butadienyl, 1,3-pentadienyl, 1,4-pentadienyl, cyclopentadienyl, and cyclohexadienyl, each of which substituents (with the exception of hydrogen and halogen) may be unsubstituted or substituted with one or more substituents selected from the group consisting of: hydrogen, halogen, hydroxyl, methoxyl, ethoxyl, propoxyl, butoxyl, carboxylic acid, amide or ester substituents, and pharmaceutically acceptable salts thereof.

Base addition salts of preferred 2-hydroxycarboxylic acids of the invention may be prepared from inorganic and organic bases. Salts derived from inorganic bases, include by way of example only, sodium, potassium, lithium, ammonium, calcium, magnesium, copper, zinc, gallium and silver salts. Salts derived from organic bases include, but are not limited to, salts of primary, secondary and tertiary amines, such as by way of example only, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

The acid addition salts of preferred 2-hydroxycarboxylic acids of the invention may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Examples of acceptable salts further include iodide, acetate, phenyl acetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, γ-hydroxybutyrate, β-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-1-sulfonate, naphthalene-2-sulfonate, p-toluenesulfonate, xylenesulfonate, tartarate, and the like.

The composition of the present disclosure may comprise a 2-hydroxycarboxylic acid of an unpreferred stereochemistry, for example L-lactic acid. The 2-hydroxycarboxylic acid of the disclosure that has the unpreferred stereochemistry is herein referred to as the “unpreferred enantiomer of a 2-hydroxycarboxylic acid”, “unpreferred enantiomer” or “unpreferred 2-hydroxycarboxylic acid”. The % of unpreferred 2-hydroxycarboxylic acid may be less than 20%. Alternatively, the composition of the present disclosure may not contain an unpreferred 2-hydroxycarboxylic acid. Thus, the composition may comprise only a 2-hydroxycarboxylic acid of a preferred stereochemistry as the 2-hydroxycarboxylic acid.

Where the unpreferred 2-hydroxycarboxylic acid is not L-lactic acid, the unpreferred 2-hydroxycarboxylic acids in accordance with the present disclosure are those in which the absolute stereochemistry of the chiral centre to which the hydroxy group of the 2-hydroxycarboxylic acid corresponds to the absolute stereochemistry of the corresponding chiral centre in L-lactic acid, in terms of the 3-dimensional orientation of the hydroxy group, as depicted below:

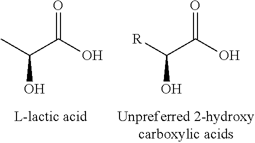

Generally, the majority of lactic acid bacteria produce a predominance of L-lactic acid. Without being held to any theory, it is believed that many bacteria have evolved to avoid or denature L-lactic acid, in order to prevent the effects of an organic acid on the bacterial membrane. Without being held to any theory, it may be that the bacteria are less able to avoid or denature the D-isomer of lactic acid, and thus it is able to have a metabolic disrupting effect.

Highly pure L-lactic acid can be produced by a broad range of microorganisms, such as bacteria, fungi, algae and cyanobacteria. On the other hand, most of D-lactic acid-producing microorganisms produce either the racemic mixture or other organic acids, such as acetic acid or succinic acid (Alexandri et al (2019) Food Tech & Biotech 57(3):293-304).

The composition of the present disclosure may comprise from 0.001% to 100% of a preferred enantiomer of a 2-hydroxycarboxylic acid, for example D-lactic acid. The composition may comprise from 0.001%, 0.01%, 0.1%, 1%, 10%, 20% 30%, 40% 50%, 60%, 70% 80% or 90% a preferred enantiomer of a 2-hydroxycarboxylic acid. The rest of the composition may comprise carriers, diluents or excipients, and/or other active agents. For example, ocular compositions may comprise from 0.001% to 1% of a preferred enantiomer of a 2-hydroxycarboxylic acid; washing and soaking, dressings and wipes, topical, inhaled or oral compositions may comprise from 0.2% to 100% of a preferred enantiomer of a 2-hydroxycarboxylic acid.

The composition of the present disclosure may comprise an unpreferred enantiomer of a 2-hydroxycarboxylic acid, wherein the % of the unpreferred enantiomer of a 2-hydroxycarboxylic acid is less than 50%, 40%, 30%, 20%, 10%, 5% or 1%. For example, the % of the unpreferred enantiomer of a 2-hydroxycarboxylic acid is less than 20%, 18%, 16%, 14%, 12%, 10%, 9%, 8%, 7%, 6%, 5%, 4%, 3%, 2% or 1%. The % of the unpreferred enantiomer of a 2-hydroxycarboxylic acid may be less than 20%.

Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid. For example, the composition may contain only D-lactic acid as the lactic acid. It has been found that a composition comprising an enantiomerically pure amount of the preferred enantiomer of a 2-hydroxycarboxylic acid has a metabolic dysregulating effect on planktonic bacteria, a composition comprising an enantiomerically pure amount of the unpreferred enantiomer of a 2-hydroxycarboxylic acid has little or no metabolic dysregulating effect and a composition comprising 1:1 ratio of the preferred enantiomer of a 2-hydroxycarboxylic acid to the unpreferred enantiomer of a 2-hydroxycarboxylic acid has a reduced metabolic dysregulating effect compared to administering the pure preferred enantiomer of a 2-hydroxycarboxylic acid or has no metabolic dysregulating effect on planktonic bacteria.

The compositions of the present disclosure optionally comprise an enantiomerically enriched amount of the preferred enantiomer of a 2-hydroxycarboxylic acid. The compositions may comprise an enantiomerically pure preferred enantiomer of a 2-hydroxycarboxylic acid.

Optionally, the concentration of a preferred enantiomer of a 2-hydroxycarboxylic acid used in a composition of the present disclosure is less than the concentration of the preferred enantiomer of a 2-hydroxycarboxylic acid required to kill bacteria.

Without being held to any theory, it is believed that the present disclosure requires a 2-hydroxycarboxylic acid with a chiral centre at position 2. It is understood that the chiral centre is at position 2 if the aliphatic chain is at least three carbons long; by this definition glycolic acid [2-hydroxy acetic acid] is not chiral as it is less than three carbons in length.

The 2-hydroxycarboxylic acid may be a hydrophilic 2-hydroxycarboxylic acid. That is, the 2-hydroxycarboxylic acid is optionally able to dissolve in water. Without being held to any theory, it is believed that the water-soluble characteristics of the 2-hydroxycarboxylic acid assist in its penetration of the planktonic bacterial membrane and metabolic dysregulating activity.

It has been found that generally 2-hydroxycarboxylic acids with seven or less carbons (including the carbons of the hydroxycarboxylic acid moiety) are more water soluble. Thus, optionally the 2-hydroxycarboxylic acid of the present disclosure comprises seven or fewer carbons. For example, 2-hydroxypropanoic acid (also known as lactic acid) comprises three carbons, 2-hydroxypentanoic acid (also known as 2-hydroxyvaleric acid) comprises five carbons and 2-hydroxycaproic acid (also known as 2-hydroxyhexanoic acid) comprises six carbons. For example, the 2-hydroxycarboxylic acid of the present disclosure comprises six or fewer carbons. In one example, the 2-hydroxycarboxylic acid of the present disclosure comprises five or fewer carbons.

Optionally, the molecular weight of the 2-hydroxycarboxylic acid is less than 800, less than 700, less than 600, less than 500. For example, the molecular weight of the 2-hydroxycarboxylic acid is less than 200, less than 180, less than 150 or less than 100. For example,

Optionally, the absolute water solubility of the 2-hydroxycarboxylic acid is more than about 0.5 g/L, 0.75 g/L or 1.0 g/L at a pH of about 5 to 10, about 9 to 6 or about 6 to 8.

Optionally, the absolute water solubility of the 2-hydroxycarboxylic acid is more than about 0.5 g/L, 0.75 g/L or 1.0 g/L at a temperature of about 20° C.

The water solubility of the 2-hydroxycarboxylic acid may be alternatively defined by its Log P: that is its Octanol-Water partition co-efficient. Log P is also known as Log K_{ow} or the n-octanol-water partition ratio. The general rule is that Log P: Octanol-Water <1.0=water soluble; Log P: Octanol-Water >1.0=hydrophobic. Optionally, the 2-hydroxycarboxylic acid of the present disclosure has a Log P: Octanol-Water <1.0. Some compounds with a Log P: <1.0 may have more than seven carbons; however, their structure allows for water solubility despite the additional carbons. For example, 2-hydroxy-2-phenylacetic acid (also known as mandelic acid) has eight carbons, but due to its cyclical structure, it has a Log P of 0.67 and is therefore water soluble.

Some 2-hydroxycarboxylic acids with special structures, such as 2-hydroxy-2-phenylacetic acid (also known as mandelic acid) that contains a six-membered ring and eight carbons in total, may still be water soluble and have a Log P: Octanol-Water <1.0. Such 2-hydroxycarboxylic acids are considered to be suitable for the present disclosure.

The 2-hydroxycarboxylic acid may have substituents, such as hydrogen, halogen, hydroxyl, methoxyl, ethoxyl, propoxyl, butoxyl, carboxylic acid, amide or ester substituents.

The 2-hydroxycarboxylic acid may be a pharmaceutically acceptable 2-hydroxycarboxylic acid. For example, the 2-hydroxycarboxylic acid is GRAS (“generally recognised as safe”), as defined under sections 201(s) and 409 of the United States Federal Food, Drug, and Cosmetic Act or equivalent regulations.

The 2-hydroxycarboxylic acid can be selected from acids, or the ester, salt, amide, or other derivatives of the group consisting of lactic acid, glycolic acid (2-hydroxyacetic acid), tartaric acid (2,3-dihydroxysuccinic acid), mandelic acid, 1-hydroxycyclohexane-1-carboxylic acid, 2-hydroxy-2-(tetrahydrofuran-2-yl)acetic acid, 2-hydroxy-2-(2-furanyl) ethanoic acid, 2-hydroxy-2-phenylpropionic acid, 2-hydroxy-2-methylpropionic acid, 2-hydroxy-2-methylbutanoic acid, 2-hydroxybutanoic acid, 2-hydroxypentanoic acid, or mixtures thereof.

The 2-hydroxycarboxylic acid may be chosen from the list comprising: 2-hydroxypropanoic acid (also known as lactic acid), 2-hydroxypentanoic acid (also known as 2-hydroxyvaleric acid), 2-hydroxybutyric acid, 2-hydroxyacetic acid (also known as glycolic acid), 2-hydroxyhexanoic acid (also known as 2-hydroxycaproic acid), and 2-hydroxy-2-phenylacetic acid (also known as mandelic acid).

The 2-hydroxycarboxylic acid may be chosen from the list comprising: D-lactic acid, 2-hydroxypentanoic acid, 2-hydroxybutyric acid, 2-hydroxthexanoic acid, (2)-hydroxyphenylacetic acid and 2-hydroxy-2-phenylacetic acid. Preferred 2-hydroxycarboxylic acids include those in Tables 1 and/or 2.

TABLE 1
Solubility of selected 2-hydroxycarboxylic acids

| | No. | | |
|---|---|---|---|
| Name | Carbons | Mw | cLogP |
| 2-hydroxyacetic acid | 2 | 76.051 | −1.0428 |
| 2-hydroxypropanoic acid | 3 | 90.078 | −0.7338 |
| 2-hydroxy-2-methylpropanoic acid | 4 | 104.105 | −0.4248 |
| 2-hydroxybutanoic acid | 4 | 104.105 | −0.2048 |
| 2,3-dihydroxysuccinic acid | 4 | 150.086 | −3.2196 |
| 2-hydroxy-2-methylbutanoic acid | 5 | 118.132 | 0.1042 |
| 2-hydroxy-3-methylbutanoic acid | 5 | 118.132 | 0.1942 |
| 2-hydroxypentanoic acid | 5 | 118.132 | 0.3242 |
| 2-hydroxy-3,3-dimethylbutanoic acid | 6 | 132.159 | 0.5932 |
| 2-hydroxy-2-methylpentanoic acid | 6 | 132.159 | 0.6332 |
| 2-hydroxy-4-methylpentanoic acid | 6 | 132.159 | 0.7232 |
| 2-hydroxy-3-methylpentanoic acid | 6 | 132.159 | 0.7232 |
| 2-hydroxyhexanoic acid | 6 | 132.159 | 0.8532 |
| 2-(furan-2-yl)-2-hydroxyacetic acid | 6 | 142.11 | −0.3216 |
| 2-hydroxy-2-(tetrahydrofuran-2-yl)acetic | 6 | 146.142 | −0.72665 |
| acid | | | |
| 1-hydroxycyclohexane-1-carboxylic acid | 7 | 144.17 | 0.8882 |
| 2-hydroxy-2,3,3-trimethylbutanoic acid | 7 | 146.186 | 0.9022 |
| 2-hydroxy-2,4-dimethylpentanoic acid | 7 | 146.186 | 1.0322 |
| 2-hydroxy-2,3-dimethylpentanoic acid | 7 | 146.186 | 1.0322 |
| 2-hydroxy-4,4-dimethylpentanoic acid | 7 | 146.186 | 1.1222 |
| 2-hydroxy-3,3-dimethylpentanoic acid | 7 | 146.186 | 1.1222 |
| 2-hydroxy-3,4-dimethylpentanoic acid | 7 | 146.186 | 1.1222 |
| 2-hydroxy-2-methylhexanoic acid | 7 | 146.186 | 1.1622 |
| 2-hydroxy-5-methylhexanoic acid | 7 | 146.186 | 1.2522 |
| 2-hydroxy-4-methylhexanoic acid | 7 | 146.186 | 1.2522 |
| 2-hydroxy-3-methylhexanoic acid | 7 | 146.186 | 1.2522 |
| 2-hydroxyheptanoic acid | 7 | 146.186 | 1.3822 |
| 2-hydroxy-2-phenylacetic acid | 8 | 152.149 | 0.5024 |
| 2-hydroxy-2-phenylacetic acid | 8 | 152.149 | 0.5024 |
| 2-hydroxy-2-phenylpropanoic acid | 9 | 166.176 | 0.8114 |
| 2-(1-hydroxyethyl)benzoic acid | 9 | 166.176 | 0.856 |

TABLE 2
Solubility of selected 2-hydroxycarboxylic acids

| | | Water solubility* |
|---|---|---|
| 2-hydroxycarboxylic acid | AKA | (Mol/L) |
| 2-Hydroxypropanoic acid | lactic acid | 3.22 |
| 2-Hydroxybutyric acid | | 1.93 |
| 2-Hydroxypentanoic acid | 2-hydroxyvaleric acid | 1.64 |
| 2-Hydroxhexanoic acid | 2-hydroxycaproic acid | 0.27 |
| 2-Hydroxy-2-phenylacetic acid | mandelic acid | 1.14 |

Optionally, the 2-hydroxycarboxylic acid has both less than seven carbons and a Log P: Octanol-Water <1.0.

Optionally, the 2-hydroxycarboxylic acid of the present disclosure is lactic acid, which ash two enantiomers. Optionally, the preferred enantiomer of a 2-hydroxycarboxylic acid is D-lactic acid, also known as (2R)-2-hydroxypropanoic acid and (R)-lactic acid.

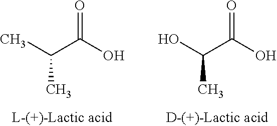

The preferred enantiomer of a 2-hydroxycarboxylic acid may be synthesised via a cell-free route (for example enzyme catalysed reactions); from Lactic Acid bacteria, including bacteria such as Lactobacillus spp. or Bifidobacterium spp; from supernatant derived from cultured bacteria such as multi-strain Gram-positive and/or Gram-negative probiotic bacterial strains; from natural sources such as 2-hydroxy-2-phenylacetic acid (mandelic acid) from almonds, olive oil and beer; or derived from other starchy biomasses.

If the preferred enantiomer of a 2-hydroxycarboxylic acid is D-lactic acid, the D-lactic acid may be synthesised via a cell-free route (for example enzyme catalysed reactions), from lactobacillus, or from supernatant derived from cultured bacteria such as multi-strain Gram-negative probiotic bacterial strains.

The D-lactic acid of the present disclosure is optionally a monomer. Thus, the D-lactic acid is optionally not polylactic acid (also known as PLA, poly D-lactic acid [PDLA], or poly L-lactic acid [PLLA]) or a similar polymer. PLA is a polymer obtained by the ring-opening polymerization of the monomer lactide (cyclic dimer of lactic acid).

### Antimicrobial Compounds

The method of the present disclosure further provides a preferred enantiomer of a 2-hydroxycarboxylic acid to co-administer in combination with an antimicrobial compound, wherein the preferred enantiomer of a 2-hydroxycarboxylic acid increases the activity of the antimicrobial compound against the planktonic bacteria. The preferred enantiomer of a 2-hydroxycarboxylic acid may be in the same composition as the antimicrobial compound, or may be in a separate composition. Planktonic bacteria may be causing an infection of a subject, an infection in or on a plant or in the soil plants are growing in, or may be present on a surface, such as a non-living surface. The planktonic bacteria may be a free-floating planktonic bacterium in a liquid or gas.

The term “antimicrobial compound” refers to compounds that can kill or reduce the vitality of bacteria. The antimicrobial compound may be an antibiotic, or agent with a different mode of antimicrobial action, such as a bacteriophage, antiseptic, saline, chlorine-based compound (such as bleach), an iodine-based compound, a copper-based compound, or heavy metal. The term includes bactericidal, bacteriostatic, antibiofilm, or antiseptic antimicrobial compounds. The antimicrobial compound may be an antibiofilm compound.

The term “antibiotic” refers to antimicrobial compounds that are active against bacteria and are used in a medical or veterinary setting, for prophylaxis or therapy. They may either kill (bactericidal) or inhibit the growth (bacteriostatic) of bacteria. The antibiotics may be naturally-derived (compounds derived from screening bacteria and fungi for antibiotic compounds; eg penicillin) or laboratory-derived (compounds derived from chemically determining targets for antibiotic compounds; eg sulfamethoxazole). The antibiotic may be a derivative of a naturally-derived or laboratory-derived compound.

If the antimicrobial compound being administered with the preferred enantiomer of a 2-hydroxycarboxylic acid of the present disclosure is an antibiotic, it may be chosen from one or more of the following classes:

| Activity | Antibiotic |
|---|---|
| Disrupting cell wall synthesis | β-lactams |
| | Penicillins |
| | Cephalosporins |
| | Carbapenems |
| | Monobactams |
| | Glycopeptides |
| Disrupting folate synthesis | Sulfonamides |
| | Trimethoprim |
| Preventing nucleic acid synthesis | DNA gyrase |
| | Quinolones |
| LPS and/or TLR4 blockers | Thalidomide |
| Disrupting cell membrane | Polymyxins |
| RNA polymerase | Rifamycins |
| Preventing protein synthesis-50S subunit | Macrolides |
| | Oxazolidinones |
| | Chloramphenicol |
| Preventing protein synthesis-30S subunit | Aminoglycosides |
| | Tetracyclines |

The term “sensitising” refers to an increase in the effect of an antimicrobial compound on the planktonic bacterium, for example by decreasing the minimum inhibitory concentration (MIC) of the antimicrobial compound. The MIC value is the lowest concentration of an antibiotic at which visible bacterial growth is inhibited. The sensitising may be through potentiation or synergy.

Preferably the planktonic bacterium is resistant to one or more antimicrobial compounds.

The term “synergy” as used herein, refers to when the 2-hydroxycarboxylic acid and the antimicrobial compound, such as an antibiotic, work together to produce an effect more potent than if each active were applied singly. For example, in the present disclosure synergy may occur when the combined effect of the 2-hydroxycarboxylic acid and the antimicrobial compound on the planktonic bacterium is greater than the sum of their effects when given separately. Synergy may occur if the planktonic bacterium is susceptible to the antimicrobial compound (for example to increase the effect of the antimicrobial compound so that less of the antimicrobial compound may be used for the same effect; by reducing the MIC etc), or if the planktonic bacterium is resistant to the antimicrobial compound (for example to increase the ability of the antimicrobial compound to act on the planktonic bacteria to such a degree that the effective use of the antimicrobial compound is now possible where it was not effective previously due to resistance; by reducing the MIC to an effective value that can be administered safely, easily and cost effectively etc).

Measuring synergy in combinations of a preferred enantiomer of a 2-hydroxycarboxylic acid and an antimicrobial compound may be accomplished using simple plate assays, where compounds are applied on separate paper disks and allowed to diffuse on agar plates inoculated with target bacteria. Synergy is indicated with enhanced killing at the interface of the diffusion zones. Quantitative measurement of synergy can be measured using the simple checkerboard strategy, where concentrations of both agents are systematically diluted to identify concentrations of both drugs that achieve the most potent interaction.

Synergy is often measured via calculation of the Fractional Inhibitory Concentration Index (FICI), where:

$$FICI = ( {MIC}_{AB} / {MIC}_{A} ) + ( {MIC}_{BA} / {MICM}_{B} )$$

MIC_{AB} is the minimum inhibitory concentration (MIC) of drug A tested in combination, MIC_{A} is the MIC of drug A tested alone, MIC_{BA} is the MIC of drug B tested in combination and MIC_{B} is the MIC of drug B tested alone. A FICI of <0.50 is defined as synergy (that is, a four-fold decrease in MIC), a FICI between 0.50 and 1.00 represents an additive effect, a FICI from 1.00 to 4.00 is defined as indifferent. A FICI above 4.00 represents antagonism.

However, determining a FICI value for the present combination of a preferred enantiomer of a 2-hydroxycarboxylic acid and an antimicrobial compound may be difficult, as the 2-hydroxycarboxylic acid does not have an antimicrobial effect itself, but rather simply increases the effect of the antimicrobial compound. In other cases, the antimicrobial compound may not have an MIC in the absence of the preferred enantiomer of a 2-hydroxycarboxylic acid. In cases where the preferred enantiomer of a 2-hydroxycarboxylic acid or the antimicrobial compound does not have a measurable MIC, the concentration of the 2-hydroxycarboxylic acid that enables determination of an MIC for the antimicrobial compound can be used. In one example, the concentration of the 2-hydroxycarboxylic acid that the reduces the MIC of the antimicrobial compound may be used as an indicator of potency. For example, a fourfold reduction in the MIC may be used as an indicator of potency (that is, a four-fold decrease in MIC). More relevant is the concentration of preferred enantiomer of a 2-hydroxycarboxylic acid that lowers the antimicrobial compound MIC in resistant bacteria to equal to, or less than, the breakpoint concentration, that is, the concentration of antimicrobial compound that defines whether a strain is resistant or sensitive. The Rescue Concentration (RC) is therefore defined as the concentration of the 2-hydroxycarboxylic acid required to lower the effective concentration of the antimicrobial compound to less than, or equal to, the breakpoint (Wright et al (2016) Trends Microbiol. 24(11):862-871).

The term “potentiation” as used herein, refers to the ability of the 2-hydroxycarboxylic acid to increase the activity of an antimicrobial compound, such as an antibiotic. For example, in the present disclosure potentiation may occur when the planktonic bacterium is resistant to an antimicrobial compound (and therefore the antimicrobial compound has no effect alone, i.e. no MIC), but on combination of the antimicrobial compound with the 2-hydroxycarboxylic acid, the response of the planktonic bacterium to the antimicrobial compound is enhanced (i.e. the antimicrobial compound has a more significant effect on the bacteria, either a bacteriostatic effect or bactericidal effect, i.e. an MIC is now determinable).

The preferred enantiomer of a 2-hydroxycarboxylic acid of the present disclosure has the ability to combat bacterial resistance via several mechanisms, such as enhancing the uptake of the antimicrobial compound, obstructing the efflux of the antimicrobial compound, disrupting signalling pathways, preventing the modification of the antimicrobial compound and/or preventing the modification of the antimicrobial compound target site.

If the dysregulation of the bacterial metabolism is suppression of metabolism by the preferred enantiomer of a 2-hydroxycarboxylic acid of the present disclosure, the metabolic suppression may work, for example, to retain the antimicrobial compound in the planktonic bacterium by slowing the efflux of the antimicrobial compound as metabolism (including the efflux pumps) is suppressed. Alternatively, the suppression may cause the antimicrobial compound to be retained in the planktonic bacterium by slowing the rate at which the antimicrobial compound is modified as metabolism (including enzyme production and activity) is suppressed.

If the dysregulation of bacterial metabolism is enhancement of metabolism by the preferred enantiomer of a 2-hydroxycarboxylic acid of the present disclosure, the metabolic enhancement may work, for example, by increasing the uptake of the antimicrobial compound. Alternative, the enhancement of metabolism may lead to increased free radical production making the bacterial cells more sensitive to the antimicrobial compound.

Optionally, the pH of the compositions of the present disclosure is from about 6.5 to 8.0, for example about 7.0 and 7.4. It has previously been found that bacteria become more resistant to antimicrobial therapy as the pH is lowered. The preferable pH assists in avoiding bacterial resistance to the effect of the preferred enantiomer of a 2-hydroxycarboxylic acid. Buffering agents may be added to adjust the pH level of the composition. Optionally, the compositions of the present disclosure contain tris(hydroxymethyl)aminomethane (TRIS, also known as THAM or tromethamine) or phosphate-buffered saline (PBS) as a buffering agent. The efficacy of the preferred enantiomer of a 2-hydroxycarboxylic acid at a pH of from about 6.5 to 8.0 is a further indication that the effect on bacterial metabolism is a result of the activity of the 2-hydroxycarboxylic acid itself on metabolic dysregulation, not just an effect of low pH killing bacteria.

Without being held to any theory, it is believed that the preferred enantiomer of a 2-hydroxycarboxylic acid of the present disclosure works by dysregulating the metabolic rate of planktonic bacteria. Dysregulation may be suppression or enhancement; or one of suppression or enhancement, followed by the other of suppression or enhancement.

Suppression of metabolism may lead to an increase in the susceptibility of the planktonic bacteria to antimicrobial compounds. The metabolic suppression is distinct from the activity of any antimicrobial compound, and may occur in the absence of any further antimicrobial compounds.

Enhancement of metabolism may lead to an increase in the susceptibility of the planktonic bacteria to antimicrobial compounds. The metabolic enhancement is distinct from the activity of any antimicrobial compound, and may occur in the absence of any further antimicrobial compounds. Nevertheless, it generally occurs in the presence of both the preferred enantiomer of a 2-hydroxycarboxylic acid and an antimicrobial compound. However, in some cases the enhancement of planktonic bacterial metabolism may occur on exposure to the preferred enantiomer of a 2-hydroxycarboxylic acid alone, without being prefaced by suppression and/or the presence of an antimicrobial compound. In such cases, the metabolic enhancement may be followed by metabolic suppression in the presence of the 2-hydroxycarboxylic acid and an antimicrobial compound.

When the metabolism of a planktonic bacteria is dysregulated, the bacteria may be subject to one or more of the following effects:
a heightened vulnerability of the bacteria to host immune system agents, other microbes, or other antimicrobial compounds including but not limited to antibiotics and bacteriophage;
a reduction in the ability of the bacteria to develop resistance to antimicrobial compounds (whether external or from the host);
a reduction in the cytoplasmic membrane potential or reduction in cytoplasmic membrane integrity; and/or
a reduction in growth of the bacteria.

The dysregulation of the bacterial metabolism can be measured as an MIC reduction (efficacy enhancement) of the antimicrobial compound, a shift in respiratory metabolism (measured by resazurin reduction), or any other measure of bacterial metabolism. All the measurements can be done either in vitro or in vivo. The degree of metabolism dysregulation must be quantifiable, preferably in a clinical setting, and the degree of metabolism dysregulation must be clinically significant. The dysregulation of the bacterial metabolism should lead to a sensitisation of the bacteria to the antimicrobial compound and/or a reduction in MIC of the antimicrobial compound, how ever the measurement of dysregulation is carried out.

Optionally, the treatment regimens of the present disclosure cause bacterial metabolic dysregulation in the form of metabolic suppression of at least two-fold as determined by for example, a resazurin reduction to resorufin. Alternatively, the metabolic dysregulation for planktonic bacteria in the form of metabolic suppression may be measured via sensitisation to antimicrobials, wherein the sensitisation is an at least a 20%, 30%, or 50% reduction in MIC or bMIC concentration for the antimicrobial.

Optionally, the treatment regimens of the present disclosure cause bacterial metabolic dysregulation in the form of metabolic enhancement of at least two-fold as determined by for example, a resazurin reduction to resorufin. Alternatively, the metabolic dysregulation for planktonic bacteria in the form of metabolic enhancement may be measured via sensitisation to antimicrobials, wherein the sensitisation is an at least a 20%, 30%, or 50% reduction in MIC or bMIC concentration for the antimicrobial.

### Planktonic Bacteria

The planktonic bacteria may be Gram-positive or Gram-negative. For example, the planktonic bacteria may be an Enterobacteriaceae, or may be chosen from the following geniuses: Bacillus spp., Clostridium spp., Campylobacter spp. Pseudomonas spp., Streptococcus spp., Actinobacillus spp., Staphylococcus spp., Escherichia spp., Acinetobacter spp., Klebsiella spp., Aeromonas spp., Enterococcus spp., Legionella spp., and Salmonella spp. Shigella spp., Gardnerella spp., Haemophilus spp., Helicobacter spp., Moraxella spp., Mycobacterium spp., Neisseria spp., Anaerococcus spp., Atopobium spp., Bacteroides spp., Leptotrichia spp., Mobiluncus spp., Peptostreptococcus spp., Prevotella spp., Sneathia spp., Sphingomonas spp., Nitrospira spp., Mycobacterium spp., or Hyphomicrobium spp. and Vibrio spp. The planktonic bacteria may be a cyanobacteria or nontuberculous mycobacteria (NTM).

The planktonic bacteria may be chosen from the bacteria comprising: Pseudomonas aeruginosa, Streptococcus pneumoniae, Streptococcus mutans, Actinobacillus pleuropneumoniae, Staphylococcus epidermidis, Escherichia coli, Staphylococcus aureus, Acinetobacter baumannii, Aeromonas hydrophila, Aeromonas salmonicida, Clostridium difficile, Enterococcus faecium, Gardnerella vaginalis, Haemophilus influenzae, Helicobacter pylori, Moraxella bovis, Moraxella catarrhalis, Mycobacterium avium, Mycobacterium intracellulare, Mycobacterium tuberculosis, Neisseria gonorrhoeae, Neisseria meningitidis, Streptococcus pyogenes, and Vibrio cholerae.

For example, Bacillus spp., Clostridium sp., and Pseudomonas spp. are often found in the gas and oil in pipelines. Sphingomonas spp., Nitrospira spp., Legionella spp., Mycobacterium spp., or Hyphomicrobium spp. are often found in the water in water pipes and the air in air conditioning units. Eubacteria, including cyanobacteria, may be present in sea water and can, once attached, form the basis of biofilm fouling on the hulls of ships.

Bacterial vaginosis may be caused by or associated with Gardnerella vaginalis and other taxonomic groups such as Anaerococcus, Atopobium, Bacteroides, Leptotrichia, Mobiluncus, Peptostreptococcus, Prevotella, Sneathia and Clostridia spp. and may be treated with the metabolic dysregulating compositions of the present disclosure.

The planktonic bacteria may be antibiotic resistant strains of the bacteria listed above. For example, the planktonic bacteria may be antibiotic resistant Pseudomonas aeruginosa or antibiotic resistant Staphylococcus aureus (e.g. MRSA).

Planktonic diarrhoeal pathogens typically infect via the ‘oral-faecal route’, that is contaminated drinking water, ice cubes in that cocktail (or soft drink), poor hand washing, etc.

The planktonic bacteria may be associated with a condition or disease such as sepsis, cystic fibrosis associated pneumonia, periodontitis, diabetic ulcers and other wound infections, otitis media, periodontitis, sinusitis, infective endocarditis, ocular infections, glue ear, dysentery, respiratory infections, sexually transmitted diseases (STDs), bacterial vaginosis, and osteomyelitis.

Alternatively, the planktonic bacteria may be associated with industrial and non-biological surfaces such as hydroelectric turbines, oil and gas pipelines, air conditioning units, soap dispensers, industrial water systems, ship hulls, etc.

In further examples, the planktonic bacteria may be associated with medical devices and instruments, such as the bags used to store blood donations and indwelling medical devices.

The planktonic bacteria may further be associated with food production and storage. For example, the planktonic bacteria may be present on food preparation surfaces, storage containers, in the water used to wash food that is not then cooked (e.g. salads) or on the foods itself.

The planktonic bacteria may further be associated with animal housing or materials and equipment associated with animal husbandry.

As used herein, “treating” or “treatment” refers to inhibiting a disease or condition, i.e., arresting or reducing its development or at least one clinical or subclinical symptom thereof. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician.

The ability of a preferred enantiomer of a 2-hydroxycarboxylic acid to dysregulate the metabolism of a planktonic bacteria may be assessed or measured using resazurin (7-Hydroxy-3H-phenoxazin-3-one 10-oxide). Bacteria undergoing aerobic respiration have a reducing cytoplasm, and under these conditions resazurin is reduced irreversibly to the highly fluorescent resorufin. The number of ‘relative fluorescent units’ (RFU) are therefore a readout of aerobic respiration/oxidative phosphorylation. The present disclosure has found that in some aspects, administration of a preferred enantiomer of a 2-hydroxycarboxylic acid to planktonic bacteria leads to a significant change (reduction or increase) in the rate of reduction of resazurin, thus the administration of a preferred enantiomer of a 2-hydroxycarboxylic acid to planktonic bacteria leads to a significant change (reduction or increase) in the rate of bacterial aerobic respiration/oxidative phosphorylation. The assay used in the present disclosure determines the minimum inhibitory concentration (MIC) of the preferred enantiomer of a 2-hydroxycarboxylic acid (rather than determining the dose/response).

Optionally, the dysregulation of metabolism induced by the preferred enantiomer of a 2-hydroxycarboxylic acid does not result in death of the planktonic bacteria. We note that it has been suggested that administration of bactericidal compounds may lead to an increase in the metabolic rate and an increase in the production of free radicals (with oxidative stress leading to cell death). In view of this, the preferred enantiomer of a 2-hydroxycarboxylic acid of the present disclosure is optionally not a bactericidal compound.

Administration of the preferred enantiomer of a 2-hydroxycarboxylic acid of the present disclosure would optionally dysregulate (suppress or enhance) the metabolism of planktonic bacteria sufficiently to allow other mechanisms (such as host defence mechanisms or bactericidal compounds) to have their effect to kill the planktonic bacteria whose metabolism has been dysregulated. Dysregulation of bacterial metabolism in one direction (suppression or enhancement) when the preferred enantiomer of a 2-hydroxycarboxylic acid is provided alone may be followed by dysregulation in the other direction (enhancement or suppression) when the preferred enantiomer of a 2-hydroxycarboxylic acid is provided in combination with an antimicrobial compound.

The planktonic bacteria being administered the preferred enantiomer of a 2-hydroxycarboxylic acid of the present disclosures may be resistant to one or more antimicrobial compounds. The resistance mechanism may take the form of one or more of the following classes of activity:
preventing entry of the antimicrobial compound into the bacterial cell;
altering the structure of the target of the antimicrobial compound;
increasing the efflux of the antimicrobial compound out of the bacterial cell; and/or
increasing the metabolism of the antimicrobial compound within the bacterial cell.

The dysregulation of metabolic activity induced by the preferred enantiomer of a 2-hydroxycarboxylic acid of the present disclosure may assist in sensitising the planktonic bacteria to an antimicrobial compound (for example in the form of an antibiotic). This dysregulation may be suppression of bacterial metabolism, which may lead to, for example, preventing efflux of the antimicrobial compound out of the bacterial cell and/or decreasing the metabolism of the antimicrobial compound within the bacterial cell. Alternatively, if the dysregulation is enhancement of bacterial metabolism induced by the preferred enantiomer of a 2-hydroxycarboxylic acid of the present disclosure, this may assist in sensitising the planktonic bacteria to an antimicrobial compound by, for example, increasing the ability of the antimicrobial compound to penetrate the planktonic bacteria, increasing the amount of free-radical in the cell and rendering it sensitive to the antimicrobial compound, and/or increasing the ability of the antimicrobial compound to bind its target despite the planktonic bacteria altering the structure of the target of the antimicrobial compound.

### Method of Dysregulation Metabolism

The present disclosure further provides a method for the dysregulation of planktonic bacterial metabolism, comprising the step of:
i. administering a preferred enantiomer of a 2-hydroxycarboxylic acid to the planktonic bacteria.

The disclosure further provides a method for the dysregulation of planktonic bacterial metabolism, comprising the step of:
i. administering a preferred enantiomer of a 2-hydroxycarboxylic acid to the planktonic bacterium
   wherein the preferred enantiomer of a 2-hydroxycarboxylic acid is in combination with an antimicrobial compound.

The present disclosure further provides a method to treat or prevent a bacterial infection by planktonic bacteria, comprising the step of:
i. administering a preferred enantiomer of a 2-hydroxycarboxylic acid to the site of infection.

The disclosure further provides a method to treat or prevent a bacterial infection by planktonic bacteria, comprising the step of:
i. administering a preferred enantiomer of a 2-hydroxycarboxylic acid to the site of infection
   wherein the preferred enantiomer of a 2-hydroxycarboxylic acid is in combination with an antimicrobial compound.

The present disclosure further provides a method to sensitise planktonic bacteria to an antimicrobial compound, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism.

The present disclosure further provides a method to sensitise planktonic bacteria to an antimicrobial compound, comprising the step of:
i. administering a preferred enantiomer of a 2-hydroxycarboxylic acid to the planktonic bacterium
   wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

Preferably the dysregulation of the planktonic bacterial metabolism is suppression of the planktonic bacterial metabolism. If the preferred enantiomer of a 2-hydroxycarboxylic acid is administered in combination with an antimicrobial compound, the dysregulation may be suppression followed by enhancement of the planktonic bacterial metabolism. However, in some cases the enhancement of planktonic bacterial metabolism may occur on exposure to the preferred enantiomer of a 2-hydroxycarboxylic acid alone, without being prefaced by suppression and/or the presence of an antimicrobial compound. In such cases, the metabolic enhancement may be followed by metabolic suppression in the presence of the 2-hydroxycarboxylic acid and an antimicrobial compound.

The term “infection” includes microbial growth on and within both biological and non-biological surfaces, tissues, liquids, and gases. For example, an infection may be the growth of Pseudomonas in lung tissue, an infection of Erwinia amylovora (fire blight) in apple and pear trees, the growth of Legionella in water pipes, or bacterial infections of blood (sepsis).

The composition of the present disclosure may comprise from 0.001% to 100% of the preferred enantiomer of a 2-hydroxycarboxylic acid. For example, ocular compositions may comprise from 0.001% to 1% of the preferred enantiomer of a 2-hydroxycarboxylic acid; washing and soaking, dressing and wipe, topical, inhaled or oral compositions may comprise from 0.2% to 100% of the preferred enantiomer of a 2-hydroxycarboxylic acid. The rest of the composition may comprise carriers, diluents or excipients, and/or other active agents. Optionally, the preferred enantiomer of a 2-hydroxycarboxylic acid is D-lactic acid.

The composition of the present disclosure may comprise an unpreferred enantiomer of a 2-hydroxycarboxylic acid, wherein the % of unpreferred enantiomer of a 2-hydroxycarboxylic acid may be less than 20%. Alternatively, the composition of the present disclosure may not contain an unpreferred enantiomer of a 2-hydroxycarboxylic acid. Thus, the composition may comprise only a preferred enantiomer of a 2-hydroxycarboxylic acid as the 2-hydroxycarboxylic acid.

The present disclosure further provides a composition comprising a preferred enantiomer of a 2-hydroxycarboxylic acid for treating or preventing a bacterial infection in a subject, wherein the bacterial infection is by planktonic bacteria, and wherein the composition dysregulates the metabolism of the planktonic bacterium.

The present disclosure further provides a composition comprising a preferred enantiomer of a 2-hydroxycarboxylic acid for treating or preventing an infection in a subject, in combination with an antimicrobial compound, wherein the infection is by planktonic bacteria, and wherein the composition dysregulates the metabolism of the planktonic bacterium.

The present disclosure further provides a composition comprising a preferred enantiomer of a 2-hydroxycarboxylic acid for treating or preventing a bacterial infection in a subject, wherein the bacterial infection is by planktonic bacteria, wherein the composition sensitises the planktonic bacterium to an antimicrobial compound, and wherein the preferred enantiomer of a 2-hydroxycarboxylic acid induces dysregulation of the planktonic bacterial metabolism.

The present disclosure further provides a composition comprising a preferred enantiomer of a 2-hydroxycarboxylic acid for treating or preventing an infection in a subject, in combination with an antimicrobial compound, wherein the infection is by planktonic bacteria, wherein the composition sensitises the planktonic bacterium to the antimicrobial compound, and wherein the preferred enantiomer of a 2-hydroxycarboxylic acid induces dysregulation of the planktonic bacterial metabolism.

The efficacy of the method of treating or preventing an infection in a subject by administering a preferred enantiomer of a 2-hydroxycarboxylic acid may be established by determining outcomes other than measuring the metabolism of the bacterium itself. For example, the efficacy may be determined by a decrease in the bacterial load of sputum samples in a subject with infective lung disease after administration of a preferred enantiomer of a 2-hydroxycarboxylic acid. In another example, the efficacy of treatment using a preferred enantiomer of a 2-hydroxycarboxylic acid against an infection in a mammary gland may be established by a reduction in visible inflammation of the outer surface of the mammary tissue, or a reduction in swelling, heat, pain and/or redness. In either the lung example or the mammary tissue example, a further test may be a microbiological assessment of biological fluids from the infection site (eg sputum, milk) to determine the microbial load. If the preferred enantiomer of a 2-hydroxycarboxylic acid is dysregulating the metabolism of the planktonic bacteria causing the infection, the microbial load of the infection site should be reduced.

### Surfaces

The preferred enantiomer of a 2-hydroxycarboxylic acid compositions of the present disclosure may be used to dysregulate the metabolism of planktonic bacteria, and optionally sensitise the planktonic bacterium to an antimicrobial compound, on both living and non-living surfaces and in living and non-living substances such as liquids and gases. For the purposes of this invention and ease of discussion, the presence of planktonic bacteria in liquids and gasses will be considered to be “on surfaces”.

Planktonic bacteria can be present on and in almost any type of surface, such as organic surfaces (living or non-living), plastic, metal, glass, soil particles, wood and food products and in living and non-living liquids and gases such as blood, air, lymph, drinking water. The present disclosure may be used to dysregulate the metabolism of planktonic bacteria in such locations.

Living surfaces that may host planktonic bacteria, the metabolisms of which may be dysregulated using the compositions of the present disclosure include: lung mucosal surfaces (particularly the lungs of subjects with cystic fibrosis or bronchiectasis); chronic wounds (including ulcers such as diabetic ulcers); skin surfaces (particular those with skin dysbiosis, acne, skin itchiness, warts, psoriasis and atopic dermatitis), inner vaginal surfaces (including in bacterial vaginosis and vulvovaginal candidiasis), gastrointestinal surfaces (such as small intestinal bacterial overgrowth, Clostridium difficile infections, stomach ulcers), the middle ear surfaces (particularly those with otitis media); periodontic surfaces (particularly those with periodontitis); sinus surfaces (particularly those infected with sinusitis); heart surfaces (particularly those with infective endocarditis), ocular surfaces (particularly those with postoperative endophthalmitis, microbial keratitis, infection crystalline keratopathy), other ocular surfaces (particularly those associated with scleral buckle insertion, conjunctival plug insertion, and lacrimal intubation) and bone surfaces (particularly those with osteomyelitis). Living substances that may host planktonic bacteria, the metabolisms of which may be suppressed using the compositions of the present disclosure include: blood, mucus, gastric fluid, tears, and lymph.

The presence of planktonic bacteria is common in food industry and represents a concern because the planktonic bacteria may be pathogenic bacteria.

Medical non-living surfaces that may host planktonic bacteria, the metabolisms of which may be suppressed using the compositions of the present disclosure include indwelling medical devices such as urinary catheters; venous catheters; intrauterine devices; prostheses (including artificial hips, knees, heart valves, etc; cochlear implants; intraocular lenses; breast implants; etc.); vascular access devices; endotracheal tubes; tracheostomies, enteral feeding tubes; wound drains; ear ventilation tubes; soap dispensers and surface medical devices such as contact lenses, orthodontic retainers and mouth guards. Medical non-living substances that may host planktonic bacteria, the metabolisms of which may be dysregulated using the compositions of the present disclosure include: oxygen or other air sources for assisting the breathing of subjects, handwashing liquids.

Industrial surfaces that may host planktonic bacteria, the metabolisms of which may be dysregulated include hydroelectric turbines, oil and gas pipelines, air conditioning units, soap dispensers, industrial water systems, ship hulls, dairy equipment etc. The planktonic bacteria may be present on animal housing or materials and equipment associated with animal husbandry. Industrial substances that may host planktonic bacteria, the metabolisms of which may be dysregulated using the compositions of the present disclosure include: lubricating oils, oxygen or other air sources, handwashing liquids.

### Plants

The preferred enantiomer of a 2-hydroxycarboxylic acid compositions of the present disclosure may be used to dysregulate the metabolism of planktonic bacteria, and optionally sensitise the planktonic bacterium to an antimicrobial compound, in and on plant tissue and in the soil plants are growing in. Many plant infections are treated using antimicrobial compounds, and resistance to the antimicrobial compounds develops in a similar manner to that encountered in infections of animal subjects. The resistance may be countered by sensitisation of the planktonic bacteria to the antimicrobial compound using the preferred enantiomer of a 2-hydroxycarboxylic acid of the present invention.

The plant may be a broadacre crop, vegetable crop, fruit crop, tree plantation, orchard, pot plant, ornamental garden, park, forest, lawn etc.

The preferred enantiomer of a 2-hydroxycarboxylic acid compositions of the present disclosure may be used to dysregulate the metabolism of planktonic bacteria on food crops, broad acre crops and vegetable and fruit crops in the field, orchard, vineyard and nursery; and on plants in domestic garden settings and pot plants and lawns, sporting ovals etc. The 2-hydroxycarboxylic acid compositions may also be used to dysregulate the metabolism of planktonic bacteria in intensive plant growth situations such as greenhouses. 2-hydroxycarboxylic acid compositions may also be used to dysregulate the metabolism of planktonic bacteria in situations with few plants, but high levels of bacteria, such as in backyards, around pig farms, chicken coops and chicken houses, and barns.

Bacterial infections of plants that may be treated by the preferred enantiomer of a 2-hydroxycarboxylic acid compositions of the present disclosure include infections caused by bacteria such as: Agrobacterium spp., Xanthomonas spp., Pseudomonas spp., Erwinia spp., Corynebacterium spp., Ralstonia spp., and Streptomyces spp. For example, the following diseases may be treated:

| Bacterial disease | Pathogen | Plants affected |
|---|---|---|
| Crown gall | Agrobacterium spp., (eg A. | many woody and herbaceous plants, |
| | tumefaciens), Allorhizobium | including fruit, vegetables and ornamental |
| | spp., Rhizobium vitis | plants. Economically significant for grapes |
| | | (vineyards), stone fruit, raspberries, roses, |
| | | nut trees, timber species, field crops e.g. |
| | | cotton, sugar beets, tomatoes, beans, alfalfa |
| Black rot | Xanthomonas campestris | Brassicas |
| Bacterial canker | Clavibacter michiganensis | Tomato; capsicum; chilli |
| Bacterial soft rot | Pseudomonas spp., Erwinia | Wide range of vegetables, including lettuce; |
| | spp. | brassicas; cucurbits; tomato; capsicum; |
| | | potato; sweet potato; carrots;herbs |
| Bacterial leaf | Xanthomonas campestris | Range of vegetables including lettuce; |
| spot/Bacterial spot | | cucurbits; tomato; capsicum |
| Bacterial wilt | Ralstonia solanacearum | Potato; tomato; capsicum; eggplant |
| Bacterial leaf | Pseudomonas syringae | Beet; spring onions; leeks; rocket; coriander |
| spot/Bacterial | | |
| spot/Bacterial blight | | |
| Bacterial blight | Pseudomonas syringae | Peas |
| Bacterial canker | Pseudomonas syringae | Kiwi fruit, cherries, stone fruit |
| Bacterial speck | Pseudomonas syringae | Tomato |
| Bacterial brown spot | Pseudomonas syringae | Beans |
| Halo blight | Pseudomonas syringae | Beans |
| Peppery leaf spot | Pseudomonas syringae | Brassicas |
| Angular leaf spot | Pseudomonas syringae | Cucurbits |
| Varnish spot | Pseudomonas spp. | Lettuce |
| Bacterial pith necrosis | Pseudomonas corrugata and | Tomato |
| | other bacteria | |
| Corky root | Rhizomonas suberifaciens | Lettuce |
| Common bacterial | Xanthomonas campestris | Beans |
| blight | | |
| Black leg | Erwinia carotovora | Potatoes |
| Leaf scorch | Xylella fastidiosa | Coffee |
| Citrus variegated | Xylella fastidiosa | Citrus |
| chlorosis | | |
| Soft rot | Dickeya spp., | Potatoes, carrots |
| | Pectobacterium spp. | |
| Fire blight | Erwinia amylovora | Apples, pears |
| Banana bacterial wilt | Xanthomonas campestris | Bananas |
| Rice bacterial blight | Xanthomonas oryzae | Rice |

### Delivery

The compositions of the present disclosure may be provided as, for instance: topical compositions such as ointments, creams, foams, adhesives or lotions, eye ointments, and eye or ear drops, or nasal sprays; impregnated dressings, patches and wipes; internal local compositions such as pessaries and suppositories; as inhaled compositions such as nebulised or dry powder compositions; as injectable formulations for intravenous and localised injections (for example into an abscess), or as oral formulations in tablet, liquid capsule, oral gel, etc form. The compositions may further be in the form of washes or soaking solutions. The compositions may further be in the form of foliar sprays or soil sprays.

The term a “therapeutically effective amount” as used herein means an amount of the composition, which when administered according to a desired dosage regimen or application regimen, is sufficient to at least partially attain the desired effect. Optionally, the desired effect is the dysregulation of the metabolism of the planktonic bacteria. However, the effect may be to delay the onset of, or inhibit the progression of, halt, partially or fully the onset or progression of an infection (including an infection of an animal, a surface or a plant) by planktonic bacteria.

The term a “preventative effective amount” as used herein means an amount of the composition, which when administered according to a desired dosage regimen or application regimen, is sufficient to at least partially dysregulate the metabolism of the planktonic bacteria.

The dysregulation of the planktonic bacterial metabolism in the presence of the preferred enantiomer of a 2-hydroxycarboxylic acid alone may be suppression or enhancement of the planktonic bacterial metabolism. After introduction of the antimicrobial compound, the state of dysregulation (suppression or enhancement) may continue, perhaps with an increase in the degree of dysregulation. However, once the planktonic bacteria is in the presence of both the preferred enantiomer of a 2-hydroxycarboxylic acid and an antimicrobial compound, the dysregulation of the planktonic bacterial metabolism may alter, to be enhancement where there was once suppression, or suppression where there was once enhancement. Alternatively, there may be no dysregulation of metabolism in the presence of the 2-hydroxycarboxylic acid alone, but significant dysregulation in the presence of the preferred enantiomer of a 2-hydroxycarboxylic acid and an antimicrobial compound, particularly where the planktonic bacterium was resistant to the antimicrobial compound in the absence of the preferred enantiomer of a 2-hydroxycarboxylic acid.

Based on the above, it will be understood by those skilled in the art that a plurality of different treatments and means of administration can be used to treat a single subject or surface. For example, subjects already receiving medications, for example as intravenous antibiotics, etc., may benefit from oral delivery, intravenous delivery, inhalation or topical application of the compositions of the present disclosure. Some subjects may receive only the present compositions comprising a preferred enantiomer of a 2-hydroxycarboxylic acid by oral administration, inhalation or topical application. For example, subjects may have symptoms of cystic fibrosis, be diagnosed as having lung infections, or have symptoms of a medical condition, which symptoms may benefit from administration to the subject of an inhaled composition comprising a preferred 2-hydroxycarboxylic acid. Alternatively, the subject may have a topical infection such as a chronic wound, ocular infection or periodontitis and the preferred enantiomer of a 2-hydroxycarboxylic acid may be administered topically. The subject may have disruptions (“dysbiosis”) within the gut, for example the small intestine, that may benefit from oral administration of the preferred enantiomer of a 2-hydroxycarboxylic acid compositions of the present disclosure. These dysbioses may contribute to enteropathogen-induced disorders, inflammatory bowel disease (IBS), Crohn's disease, ulcerative colitis or colorectal cancer.

The compositions of the present disclosure may be used on plants, to treat plant infections on and in plant tissue, and in the soil plants are grown in.

The compositions of the present disclosure may be used on non-living surfaces, such as in in the form of moist wipes, as washes or soaking solutions for non-living surfaces such as indwelling devices and water pipes, and as cleaning compositions for pipes, food preparation machines and medical equipment. If the composition is being applied to a non-living surface, the 2-hydroxycarboxylic acid wipe, wash or soak may be followed by washing with conventional detergents, UV sterilization or bleach.

The compositions of the disclosure may also be used diagnostically. In an embodiment, for example, a subject may receive a dose of a composition of the disclosure as part of a procedure to diagnose infections associated with planktonic bacteria (such as lung infections), wherein one of more of the subject's symptoms improves in response to the composition.

In accordance with certain embodiments, the compositions are administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present disclosure. Optionally, the composition of the treatment regimen is administered to the subject between 1 and 5 times per day, for example once or twice per day.

If the administration is to a non-living surface, the compositions may be applied to the surface regularly until removal or disruption of the planktonic bacteria is obtained, optionally by dysregulation of the metabolism of the planktonic bacteria. In one preferred embodiment, the composition is applied to the surface every hour, every 2 hours, every 3 hours, once daily, twice daily, three times daily, four times daily, five times daily, once weekly, twice weekly, once fortnightly and once monthly. However, other application schedules may be utilized in accordance with the present disclosure. The compositions may be applied to an indwelling device before insertion, and after removal.

Optionally, the compositions of the present disclosure are administered, for example, orally, topically (ophthalmically, buccally and sublingually, rectally, vaginally, intranasally) or by aerosol administration. If the compositions are to be delivered to non-living surfaces, the compositions may be administered as a wash, soaking solution or wipe. The mode of administration or application is optionally suitable for the form in which the composition has been prepared. The mode of administration for the most effective response may be determined empirically and the means of administration or application described below are given as examples, and do not limit the method of delivery of the composition of the present disclosure in any way.

The compositions of the present disclosure may optionally include pharmaceutically acceptable nontoxic excipients and carriers. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The compositions of the present disclosure may be selected from the group consisting of: an immediate release composition, a delayed release composition, a controlled release composition and a rapid release composition.

The compositions of the present disclosure may further comprise an anti-inflammatory agent (such as a corticosteroid).

The compositions described herein may be formulated as oil-in-water emulsions, and/or a water-in-oil emulsions. In such a composition, the immediate release dosage form may be in the continuous phase, and the delayed release dosage form may be in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Optionally, the composition contains water, i.e. it is aqueous. In another embodiment, the composition does not comprise a preservative. In one embodiment, the compositions described herein may be aqueous and contain 0-90% water. In other embodiments, the aqueous compositions described herein may contain 20-80% water. In still other embodiments, aqueous compositions may contain 50-70% water. The water may further comprise water that is plain, distilled, sterile, demineralized or deionized. Alternatively, the composition may be non-aqueous and contain no water, or negligible amounts of water (e.g. below 1%, below 0.1%, below 0.01%).

Pharmaceutical or veterinary compositions may be formulated according to conventional pharmaceutical or veterinary practices (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pennsylvania, USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the preferred enantiomer of a 2-hydroxycarboxylic acid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the disclosure may be formed from standard vesicle-forming lipids, generally including neutral and negatively charged phospholipids and a sterol, such as cholesterol.

### Washing and Soaking Compositions

The compositions of the present disclosure comprising a preferred enantiomer of a 2-hydroxycarboxylic acid can be used to wash and/or soak devices such as indwelling medical devices or water piping systems. Washing or soaking of such devices with the preferred enantiomer of a 2-hydroxycarboxylic acid containing compositions will assist in dysregulating the metabolism of any planktonic bacteria present on the device surface and thus assist in sterilising or cleaning the device, by rendering the microorganisms present easier to remove and/or kill. The treatment optionally dysregulates the metabolism sufficiently to increase the effect of antimicrobial compounds (such as bactericidal, bacteriostatic, antibiofilm or antiseptic antimicrobial compounds).

In one form, the compositions used for washing or soaking may comprise from 0.1% to 100% of a preferred enantiomer of a 2-hydroxycarboxylic acid. The rest of the composition may comprise carriers, diluents or excipients, and/or other active agents.

In one form, the compositions used for washing or soaking may comprise an unpreferred enantiomer of a 2-hydroxycarboxylic acid, wherein the % of the unpreferred enantiomer of a 2-hydroxycarboxylic acid may be less than 20%. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

### Dressings and Wipes

The compositions of the present disclosure comprising a preferred enantiomer of a 2-hydroxycarboxylic acid can be provided in the form of impregnated dressings or bandages, or moist wipes. The dressings may be in a form that can be applied to a wound or topical surface that may be infected with planktonic bacteria, and may be left in situ for a period of hours, days or weeks. Wipes may be used to reduce the presence of planktonic bacteria on, for example, wounds or mucosal surfaces such as vaginal or rectal surfaces or on non-living surfaces. The wipes may be used hourly, daily, weekly or as needed. For example, a wipe may be used after each urination or bowel movement to dysregulate the metabolism of planktonic bacteria present on vaginal or rectal mucosal tissues. Alternatively, a wipe may be used on a food preparation surface prior to use.

In one form, the compositions used in dressings or wipes may comprise from 0.2% to 100% of a preferred enantiomer of a 2-hydroxycarboxylic acid. The rest of the composition may comprise carriers, diluents or excipients, and/or other active agents.

In one form, the compositions used in dressings or wipes may comprise an unpreferred enantiomer of a 2-hydroxycarboxylic acid, wherein the % of the unpreferred enantiomer of a 2-hydroxycarboxylic acid may be less than 20%. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

### Inhaled Delivery

The compositions of the present disclosure may be delivered via nebulised delivery or other aerosol delivery device. This is particularly suitable for planktonic bacterial related respiratory and ear-nose and throat (ENT) diseases.

Optionally, the composition is administered to the subject in need between about once per day to about six times per day, for example about once or twice per day.

Alternatively, the composition may be administered to the subject in need via continuous inhalation, via a nebuliser. The nebulised composition may be delivered for 24 hours, 12 hours, 8 hours, 6 hours, 4 hours, 2 hours or 1 hour, and each of these deliveries (apart from the 24 and 12 hour) may be repeated several times within a 24-hour period.

In one form, the compositions delivered via nebuliser or aerosol delivery may comprise from 0.2% to 100% of a preferred enantiomer of a 2-hydroxycarboxylic acid. The rest of the composition may comprise carriers, diluents or excipients, and/or other active agents.

In one form, the compositions delivered via nebuliser or aerosol delivery may comprise an unpreferred enantiomer of a 2-hydroxycarboxylic acid, wherein the % of the unpreferred enantiomer of a 2-hydroxycarboxylic acid may be less than 20%. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

A subject may typically receive a dose of from 1 to 500 mg/mL of the preferred enantiomer of a 2-hydroxycarboxylic acid, ±20% or ±10%. The dose may be delivered over varying periods of time, for example in a single puff (seconds), or over several minutes to hours. The dose may be delivered over 5 min, 10 min, 15 min, 20 min, 30 min, 45 min, 60 min, 120 min, or longer. This dose will typically be administered either by nebuliser or by at least one, preferably several, “puffs” from an aerosol device. For example, a subject may receive between 1 mg/mL and 500 mg/mL of the preferred enantiomer of a 2-hydroxycarboxylic acid in a single dose per day, or in several doses over a day.

The total dose per day is optionally administered at least once per day, but may be divided into two or more doses per day. Some subjects may benefit from a period of “loading” the subject with the preferred enantiomer of a 2-hydroxycarboxylic acid with a higher dose or more frequent administration over a period of days or weeks, followed by a reduced or maintenance dose. As cystic fibrosis, COPD etc., are typically chronic conditions, subjects are expected to receive such therapy over a prolonged period of time.

A wide range of mechanical devices designed for pulmonary delivery of therapeutic products exist, including but not limited to nebulizers, metered-dose inhalers, and powder inhalers, all of which are familiar to those skilled in the art. Some specific examples of commercially available devices suitable for the practice of this disclosure are the Ultravent nebulizer, manufactured by Mallinckrodt, Inc., St. Louis, Missouri; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Massachusetts.

All such devices require the use of compositions suitable for the dispensing of the preferred enantiomer of a 2-hydroxycarboxylic acid. Typically, each composition is specific to the type of device employed and may involve the use of an appropriate propellant material, in addition to the usual diluents, adjuvants and/or carriers useful in therapy.

Regardless of the form of the drug composition, it is preferable to create droplets or particles for inhalation in the range of about 0.1 μm to 12 μm, or about 0.25 μm to 6 μm, optionally 1 μm to 6 μm, for example about 2 μm to 4 μm. Alternatively, the particles may be 0.1 μm to 1.0 μm, 0.2 μm to 0.9 μm, 0.3 μm to 0.8 μm, 0.4 μm to 0.7 μm, or 0.5 μm. By creating inhaled particles which have a relatively narrow range of size, it is possible to further increase the efficiency of the drug delivery system and improve the repeatability of the dosing. Thus, it is preferable that the particles not only have a size in the range of 0.1 μm to 12 μm or 2 μm to 6 μm or about 3 to 4 μm but that the mean particle size be within a narrow range so that 80% or more of the particles being delivered to a subject have a particle diameter which is within +20% of the average particle size, or +10% of the average particle size, for example ±5% of the average particle size.

“Particle size” is a notion introduced for comparing dimensions of solid particles, liquid particles (droplets). For droplets and aerosols, terms such as “aerodynamic diameter” and “mass median aerodynamic diameter” (MMAD) are used.

“Aerodynamic diameter” is the diameter of a unit-density sphere having the same terminal settling velocity as the particle in question. It is used to predict where in the respiratory tract such particles will deposit.

“Mass Median Aerodynamic Diameter” is the geometric mean aerodynamic diameter. Fifty percent of the particles by weight will be smaller than the MMAD, 50% will be larger.

During particle sizing experiment, the suspensions contain innumerable number of particles of varying sizes in motion. When the particle-sizing machine analyses these particles, it forms a particle distribution curve, which covers the entire particle size range starting from the smallest particle, which could be 1 nm to the largest, which could be 100 μm. In the particle size distribution curve, a cumulative frequency is calculated for the particles. Diao refers to that particular particle diameter where 10% of the particles in the suspension have a smaller diameter or equal diameter as that of the particular particle diameter.

D₅₀: Similar to the D₁₀, D₅₀ is the cut off diameter for 50% of the particle population in the composition and refers to that particular particle diameter where 50% of the particles in the suspension have a smaller diameter or equal diameter as that of the particular particle diameter.

D₉₀: D₉₀ is the cut off diameter for 90% of the particle population in the composition and refers to that particular particle diameter where 90% of the particles in the suspension have a smaller diameter or equal diameter as that of the particular particle diameter.

The term “respiratory tract” shall be taken to mean a system of cells and organs functioning in respiration, in particular the organs, tissues and cells of the respiratory tract include, lungs, nose, nasal passage, paranasal sinuses, nasopharynx, larynx, trachea, bronchi, bronchioles, respiratory bronchioles, alveolar ducts, alveolar sacs, alveoli, pneumocytes (type 1 and type 2), ciliated mucosal epithelium, mucosal epithelium, squamous epithelial cells, mast cells, goblet cells, neutrophils, macrophages and intraepithelial dendritic cells.

In one form of the disclosure, the method of dysregulation of the metabolism of planktonic bacteria in the lung of a subject comprises administering a therapeutically effective or preventative effective concentration of the preferred enantiomer of a 2-hydroxycarboxylic acid, in the form of one or more doses of from 1 to 1000 mg/mL, for example from 5 to 500 mg/mL.

In one form of the disclosure, the method of dysregulation of the metabolism of planktonic bacteria in the lung of a subject comprises administering a therapeutically effective concentration of an inhaled composition comprising a preferred enantiomer of a 2-hydroxycarboxylic acid, in the form of one or more doses of from 1 to 1000 mg/kg/day, for example from 6 to 600 mg/kg/day.

In one form of the disclosure, the method of dysregulation of the metabolism of planktonic bacteria in the lung of a subject by administering a preventative effective concentration of an inhaled composition comprising a preferred enantiomer of a 2-hydroxycarboxylic acid, in the form of one or more doses of from 1 to 1000 mg/mL, for example from 5 to 500 mg/mL.

The compositions of the disclosure may be administered to a subject using a disposable package and portable, hand-held, battery-powered device, such as the AERx device (U.S. Pat. No. 5,823,178, Aradigm, Hayward, Calif.). Alternatively, the compositions of the instant disclosure may be carried out using a mechanical (non-electronic) device. Other inhalation devices may be used to deliver the compositions including conventional jet nebulizers, ultrasonic nebulizers, soft mist inhalers, dry powder inhalers (DPIs), metered dose inhalers (MDIs), condensation aerosol generators, and other systems.

For use as aerosols, the compounds of the present disclosure in solution or suspension may be packaged in a pressurized aerosol container together with suitable propellants, for example, hydrocarbon propellants like propane, butane, or isobutane with conventional adjuvants. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

An aerosol may be created by forcing drug through pores of a membrane which pores have a size in the range of about 0.25 to 6 μm (U.S. Pat. No. 5,823,178). When the pores have this size the particles which escape through the pores to create the aerosol will have a diameter in the range of 0.5 to 12 μm. Drug particles may be released with an air flow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the composition keeping in mind that an object of some embodiments is to provide aerosolized particles having a diameter in the range of about 0.5 to 12 μm.

### Topical Delivery

The compositions of the present disclosure may be delivered topically. The topical administration may comprise the administration of the therapeutically effective amount of a preferred enantiomer of a 2-hydroxycarboxylic acid directly to a dermal, ocular or mucosal surface of the subject. Optionally, the preferred enantiomer of a 2-hydroxycarboxylic acid is applied topically to the skin, mucosal membranes (oral, nasal, vaginal, rectal) or eye of the subject. The use may comprise administering a therapeutically effective amount of the preferred enantiomer of a 2-hydroxycarboxylic acid to the skin, mucosal membranes (oral, nasal, vaginal, rectal) or eye of a subject.

Compositions of the disclosure may be administered topically. Therefore, contemplated for use herein are compositions adapted for the direct application to the skin.

In one form, the compositions used for topical delivery may comprise from 0.2% to 100% of the preferred enantiomer of a 2-hydroxycarboxylic acid. The rest of the composition may comprise carriers, diluents or excipients, and/or other active agents.

In one form, the compositions used for topical delivery may comprise an unpreferred enantiomer of a 2-hydroxycarboxylic acid, wherein the % of the unpreferred enantiomer of a 2-hydroxycarboxylic acid may be less than 20% of the total 2-hydroxycarboxylic acid content. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the disclosure may be provided in the form of a water-based composition or ointment which is based on organic solvents such as oils. Alternatively, the compositions of the disclosure may be applied by way of a liquid spray comprising film forming components and at least a solvent in which the preferred enantiomer of a 2-hydroxycarboxylic acid is dispersed or solubilised.

The composition of the disclosure may be provided in a form selected from the group comprising, but not limited to, a rinse, a shampoo, a lotion, a gel, a leave-on preparation, a wash-off preparation, and an ointment.

Various topical delivery systems may be appropriate for administering the compositions of the present disclosure depending up on the preferred treatment regimen. Topical compositions may be produced by dissolving or combining the preferred enantiomer of a 2-hydroxycarboxylic acid of the present disclosure in an aqueous or non-aqueous carrier. In general, any liquid, cream, or gel or similar substance which does not appreciably react with the compound or any other of the active ingredients and which is non-irritating, is suitable and may be introduced into the composition. Appropriate non-sprayable viscous, semi-solid or solid forms can also be employed that include a carrier compatible with topical application and have a dynamic viscosity that is greater than water.

Suitable compositions are well known to those skilled in the art and include, but are not limited to, solutions, suspensions, emulsions, creams, gels, ointments, powders, liniments, salves, aerosols, transdermal patches, etc., which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the preferred 2-hydroxycarboxylic acid is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface-active agent, such as sodium lauryl sulfite; hydrophilic colloids, such as acacia colloidal clays, vegetable gum such as veegum and the like. Upon formation of the emulsion, the preferred enantiomer of a 2-hydroxycarboxylic acid can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the preferred enantiomer of a 2-hydroxycarboxylic acid is added to the composition at the desired concentration at a point preceding addition of the gelling agent.

Topically delivered compositions for application to mucosal surfaces such as oral, vaginal, nasal or rectal mucosal surfaces, or skin wounds, may comprise from 5% to 100% of the preferred enantiomer of a 2-hydroxycarboxylic acid. The rest of the composition may comprise carriers, diluents or excipients, and/or other active agents. In one aspect the higher dosages may be used in medically supervised situations, and the lower dosages may be used for non-life-threatening wounds treated at home.

In one form, the compositions used for application to mucosal surfaces and/or skin wounds may comprise an unpreferred enantiomer of a 2-hydroxycarboxylic acid, wherein the % of the unpreferred enantiomer of a 2-hydroxycarboxylic acid may be less than 20%. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

Compositions of the disclosure may be administered via topical ocular delivery. Optionally, the ocular composition comprises from 0.001% to 1% of the preferred enantiomer of a 2-hydroxycarboxylic acid.

Ocular delivery encompasses delivery to the sclera, retina, intraocular fluid, tissue surrounding the eyeball. For example, the delivery may be topical delivery (creams, gels, ointments, sprays, eye drops), intraocular implant or other means.

Ocular delivery may also comprise injecting the preferred enantiomer of a 2-hydroxycarboxylic acid into the sclera, intraocular space or into the area behind the eye. Compositions suitable for ocular injection optionally include sterile aqueous solutions (where water-soluble) or dispersions and sterile powders for the extemporaneous preparation of sterile injectable solutions or dispersion. Alternatively, the compounds of the disclosure are, in certain aspects encapsulated in liposomes and delivered in injectable solutions to assist their transport across cell membrane. Alternatively, or in addition, such preparations contain constituents of self-assembling pore structures to facilitate transport across the cellular membrane.

### Orally Delivered Compositions

Compositions of the disclosure may be administered via oral delivery.

In one form, the compositions delivered orally may comprise from 5% to 100% of the preferred enantiomer of a 2-hydroxycarboxylic acid. The rest of the composition may comprise carriers, diluents or excipients, and/or other active agents.

In one form, the compositions delivered orally may comprise an unpreferred enantiomer of a 2-hydroxycarboxylic acid, wherein the % of the unpreferred enantiomer of a 2-hydroxycarboxylic acid may be less than 20%. Alternatively, the composition of the present disclosure may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

A subject may typically receive a dose of from 0.1 mg/kg/day to 2 g/kg/day of a preferred enantiomer of a 2-hydroxycarboxylic acid, 20% or +10%.

Despite the varying pH of the digestive system (acidic in the stomach, alkaline in the small intestine), the efficacy of the preferred enantiomer of a 2-hydroxycarboxylic acid against planktonic bacteria remains. The pH of the surrounding fluids does not affect the metabolism dysregulating capabilities of the preferred enantiomer of a 2-hydroxycarboxylic acid. This is because the 2-hydroxycarboxylic acid remains stable in the gastrointestinal tract and has shown to maintain its functionality, as observed in many studies investigating the acid resistance of lactic acid bacteria (such as Wang et al, 2018, Archives of Microbiology, 200, 195-201) and lactic acid acidosis a metabolic acidosis that occurs from excessive fermentation in the gastrointestinal tract.

The oral compositions of the present disclosure may be delivered with an immune boosting or modulating agents. Examples of immune boosting or modulating agents include specific immunostimulants (such as vaccines and antigens) and non-specific agents such as honeybee products (including propolis and honey), pro- and pre-biotics, hormones, vitamins (vitamin c, vitamin d), minerals (zinc oxide), antioxidants (including glutathione), interferons (including INF-alpha), interleukins (including interleukin 10), colchicine, thalidomide and imiquimod. The immune booster may be delivered at the same time as the composition comprising a preferred enantiomer of a 2-hydroxycarboxylic acid, or after the composition comprising a preferred enantiomer of a 2-hydroxycarboxylic acid.

### Plant Delivery

Application of compositions comprising a preferred enantiomer of a 2-hydroxycarboxylic acid to plants and/or soil may be in the form of a spray applied using a pump-pack, hand spray or boom spray; applied using a hose or jet or crop-duster equipped aircraft; and through watering systems, i.e., trickle, mini sprinklers, flood irrigation, etc. The composition may further be injected into plants or trees.

### Subject

If the preferred enantiomer of a 2-hydroxycarboxylic acid is being administered to a subject, the subject may be any subject capable of infection by a bacteria, virus, fungi or archaea. The subject may be any animal, including fish, crustacea, molluscs, mammals, reptiles or birds. Optionally, the subject is a mammal selected from the group comprising human, canine, avian, porcine, bovine, ovine, equine, rodent, mustelid, lagomorph and feline. The subject may be a companion animal, domestic animal, or animal of aquacultural or agricultural importance. In one example, the subject is human.

### Excipients

The above-exemplified forms of the compositions described herein can be manufactured by methods well known to one of skill in the art of composition science. Additionally, the compositions described herein may include other optional excipients to aid in the manufacturing and/or administration of the compositions described herein. In one embodiment, the composition further comprises one or more pharmaceutically or physiologically acceptable carriers, diluents or excipients. Non-limiting examples of such excipients are well known in the art and include flavourings, colorants, palatants, antioxidants, viscosity modifying, tonicity agents, drug carriers, sustained-release agents, comfort-enhancing agents, emulsifiers, solubilizing aids, lubricants, binding agents and other stabilizing agents to aid in the manufacturing and/or administration of the compositions.

Compositions of the disclosure are intended for pharmaceutical or veterinary use, or for use in cleaning equipment such as medical devices, water pipes or food preparation equipment.

Optionally, the present composition is sterile. In addition to or in lieu of sterilization, the compositions of the present disclosure may contain a pharmaceutically acceptable preservative to minimize the possibility of microbial contamination. In another embodiment, the composition of the present disclosure is stable. A pharmaceutically acceptable preservative may be used in the present compositions to increase the stability of the compositions. It should be noted, however, that any preservative must be chosen for safety, as the treated tissues may be sensitive to irritants. Preservatives suitable for use herein include, but are not limited to, those that protect the solution from contamination with pathogens, including phenylethyl alcohol, benzalkonium chloride or benzoic acid, or benzoates such as sodium benzoate and phenylethyl alcohol. In certain embodiments, the compositions herein comprise from about 0.001% to about 10.0% w/w of benzalkonium chloride, or from about 0.01% v/w phenylethyl alcohol. Preserving agents may also be present in an amount from about 0.001% to about 1%, for example about 0.002% to about 0.02%, or about 0.02% w/w.

The compositions provided herein may also comprise from about 0.001% to about 90%, or about 0.001% to about 50%, or about 0.001% to about 25%, or about 0.001% to about 10%, or about 0.001% to about 1% of one or more emulsifying agent, wetting agent, or suspending agent. Such agents for use herein include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan monooleate, polyoxyethylene (20) sorbitan monopalmitate, polyoxyethylene (20) sorbitan monostearate; lecithins; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-21 actylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract.

The compositions of the present disclosure may comprise from about 0.001% to about 5% by weight of a humectant to inhibit drying of the mucous membrane and to prevent irritation. Any of a variety of pharmaceutically acceptable humectants can be employed, including sorbitol, propylene glycol, polyethylene glycol, glycerol or mixtures thereof, for example.

The disclosure encompasses variations on the above composition, as the amounts of the respective compounds may vary by ±5%, ±7.5%, ±10%, ±15%, ±17.5%, or ±20%.

The present disclosure encompasses compositions wherein the relative proportions of the active ingredient and/or each excipient independently vary from those specified above. In one form of the disclosure, the relative proportions of the active ingredient and/or each excipient independently vary by up to 50% from those specified above. In one form of the disclosure, the relative proportions of the active ingredient and/or each excipient independently vary by up to 40% from those specified above. In one form of the disclosure, the relative proportions of the active ingredient and/or each excipient independently vary by up to 30% from those specified above. In one form of the disclosure, the relative proportions of the active ingredient and/or each excipient independently vary by up to 20% from those specified above. In one form of the disclosure, the relative proportions independently vary by up to 10% from those specified above. In one form of the disclosure, the relative proportions of the active ingredient and/or each excipient independently vary by up to 5% from those specified above. In one form of the disclosure, the relative proportions independently vary by up to 10% from those specified above. In one form of the disclosure, the relative proportions of the active ingredient and/or each excipient independently vary by up to 2% from those specified above.

As would be understood by a person skilled in the art, the sum of the percentages of the excipients and the active cannot exceed 100%, and the variations described above are subject to this limitation. As would be understood by a person skilled in the art, the sum of the percentages of the excipients and the active may be less than 100%, as forms of the disclosure include components other than those specified.

The variation described above is a percentage variation of a relative proportion. By way of example, a 20% variation of the relative proportion of a component (excipient or active) that is specified at 1% means that the relative proportion of that component may be 0.8-1.2%.

### Inhaled Delivery Excipients

Compositions suitable for use with a nebulizer, either jet or ultrasonic, will typically comprise the preferred enantiomer of a 2-hydroxycarboxylic acid suspended in water or non-aqueous solvent. The composition may also include a buffer and a simple sugar (e.g., for stabilization and regulation of osmotic pressure). The nebulizer composition may also contain a surfactant, to reduce or prevent surface induced aggregation of the preferred enantiomer of a 2-hydroxycarboxylic acid caused by atomization of the solution in forming the aerosol. Also, the use of liposomes, microcapsules or microspheres, inclusion complexes, or other types of carriers is contemplated.

Compositions for use with a metered dose inhaler device will generally comprise a finely divided powder containing the preferred enantiomer of a 2-hydroxycarboxylic acid suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the preferred enantiomer of a 2-hydroxycarboxylic acid and may also include a bulking agent, such as lactose, sorbitol, sucrose, or mannitol in amounts which facilitate dispersal of the powder from the device, e.g., 50 to 90% by weight of the composition. The preferred enantiomer of a 2-hydroxycarboxylic acid should most advantageously be prepared in particulate form with an average particle size of less than 10 microns, for example 0.5 to 5 microns, for most effective delivery to the distal lung.

In one embodiment, the composition of the present disclosure may comprise a preservative, suspending agent, wetting agent, tonicity agent and/or diluent. The compositions provided herein may comprise from about 0.01% to about 90%, or about 0.01% to about 50%, or about 0.01% to about 25%, or about 0.01% to about 10%, or about 0.01% to about 5% of one or more pharmacologically suitable suspending fluids which is physiologically acceptable upon administration by inhalation. Pharmacologically suitable fluids for use herein include, but are not limited to, polar solvents, including, but not limited to, compounds that contain hydroxyl groups or other polar groups. Solvents include, but are not limited to, water or alcohols, such as ethanol, isopropanol, and glycols including propylene glycol, polyethylene glycol, polypropylene glycol, glycol ether, glycerol and polyoxyethylene alcohols. Polar solvents also include protic solvents, including, but not limited to, water, aqueous saline solutions with one or more pharmaceutically acceptable salt(s), alcohols, glycols or a mixture there of. In one alternative embodiment, the water for use in the present compositions should meet or exceed the applicable regulatory requirements for use in inhaled drugs.

In one embodiment, the compositions described herein may be aqueous and contain 0-90% water. In other embodiments, the aqueous compositions described herein may contain 20-80% water. In still other embodiments, aqueous compositions may contain 50-70% water. The water may further comprise water that is plain, distilled, sterile, demineralized or deionized. Alternatively, the composition may be non-aqueous and contain no water, or negligible amounts of water (e.g. below 1%, below 0.1%, below 0.01%).

The composition of the present disclosure may further comprise an adjuvant, such as: a bronchodilator, an anti-inflammatory agent, a surfactant, aspirin, or ethyl alcohol.

Bronchodilators optionally used in the compositions of the disclosure include but are not limited to β₂-adrenergic receptor agonists (such as albuterol, bambuterol, salbutamol, salmeterol, formoterol, arformoterol, levosalbutamol, procaterol, indacaterol, carmoterol, milveterol, procaterol, terbutaline, and the like), and antimuscarinics (such as trospium, ipratropium, glycopyrronium, aclidinium, and the like). Combinations of drugs may be used.

Anti-inflammatories that may optionally be used in the compositions of the disclosure include but are not limited to inhaled corticosteroids (such as beclometasone, budesonide, ciclesonide, fluticasone, etiprednol, mometasone, and the like), leukotriene receptor antagonists and leukotriene synthesis inhibitors (such as montelukast, zileuton, ibudilast, zafirlukast, pranlukast, amelubant, tipelukast, and the like), cyclooxygenase inhibitors (such as ibuprofen, ketoprofen, ketorolac, indometacin, naproxen, zaltoprofen, lornoxicam, meloxicam, celecoxib, lumiracoxib, etoricoxib, piroxicam, ampiroxicam, cinnoxicam, diclofenac, felbinac, lornoxicam, mesalazine, triflusal, tinoridine, iguratimod, pamicogrel, and the like). Combinations of drugs may be used. Aspirin may also be added to act as an anti-inflammatory agent.

Surfactants covered by the disclosure include but are not limited to synthetic surfactant (Exosurf®), dipalmitoylphosphatidylcholine and oleic acid. Combinations of drugs may be used. Antioxidants such as glutathione and vitamin E, zinc and zinc salts of EDTA, may be added.

Ethyl alcohol vapour acts as an anti-foaming agent in the lungs and makes sputum more liquid, which can aid breathing and reduce lung oedema. Ethanol may be added to the compositions of the present disclosure at between 0.5% and 60%, for example between 1 and 40%, 1 and 20%, or 1 and 10%. The ethanol may be added at 1%, 2%, 3%, 4%, 5%, 6%, 7%, 8%, 9%, 10%, 15%, 20%, 25%, 30%, 35%, 40%, 45%, 50%, 55% or 60%.

The disclosure also relates to the use of a preferred enantiomer of a 2-hydroxycarboxylic acid in combination with other drugs given via inhalation. These other drugs may include a nucleotide sequence which may be incorporated into a suitable delivery vector such as a plasmid or viral vector. The other drug may be a therapeutic nucleotide sequence (DNA, RNA, siRNA), enzymes to reduce the viscoelasticity of the mucus such as DNase and other mucolytic agents, chemicals to upregulate the chloride ion channel or increase flow of ions across the cells, nicotine, P2Y2 agonists, elastase inhibitors including α-1 antitrypsin (AAT), N-acetylcysteine, antibiotics and cationic peptides, such as lantibiotics, and specifically duramycin, short-acting bronchodilators (e.g., β2-adrenergic receptor agonists like albuterol or indacaterol), M3 muscarinic antagonists (e.g., ipatropium bromide), K⁺-channel openers, long-acting bronchodilators (e.g., formoterol, salmeterol), steroids (e.g., budesonide, fluticasone, triamcinolone, beclomethasone, ciclesonide, etc.), xanthines, leukotriene antagonists (e.g., montelukast sodium), phosphodiesterase 4 inhibitors, adenosine receptor antagonists, other miscellaneous anti-inflammatories (e.g., Syk kinase inhibitors (AVE-0950), tryptase inhibitors (AVE-8923 & AVE-5638), tachykinin antagonists (AVE-5883), inducible nitric oxide synthase inhibitors (GW-274150) and others), transcription factor decoys, TLR-9 agonists, antisense oligonucleotides, siRNA, DNA, CGRP, lidocaine, inverse 32-agonists, anti-infective oxidative therapies, cytokine modulators (e.g., CCR3 receptor antagonists (GSK-766994, DPC-168, AZD-3778), TNF-α production inhibitors (LMP-160 & YS-TH2), and IL-4 antagonists (AVE-0309)), small molecule inhibitors of IgE, cell adhesion molecule (CAM) inhibitors, small molecules targeting the VLA4 receptor or integrin.alpha.4.beta.1 (e.g., R-411, PS-460644, DW-908e, & CDP-323), immunomodulators including those that block T-cell signalling by inhibition of calcineurin (Tacrolimus), heparin neutralizers (Talactoferrin a), cytosolic PLA2 inhibitors (Efipladib), or combinations thereof. If the subject in need has CF, then they may also be administered standard medications such as ivacaftor, pulmozyme, mannitol, or other approved drugs according to standard practise, in combination with the compositions of the present disclosure.

### Topical Delivery Excipients

The compositions of the present disclosure comprising a preferred enantiomer of a 2-hydroxycarboxylic acid may be delivered to topical surfaces. For example, the topical formulations may be for topical application to the skin, mucosal membranes (oral, nasal, vaginal, rectal) or eye of the subject.

The compositions of the present disclosure may contain water (aqueous) or may be non-aqueous.

The composition of this disclosure may also include minor amounts of conventional additives such as viscosity modifiers, for example xanthan gum, and preservatives, such as phenoxyethanol or benzyl alcohol, including mixtures thereof. For some therapeutic agents it may be necessary to incorporate buffering agents to maintain a suitable pH.

Suitable preservatives for use in such a composition or medicament include, for example, phenoxyethanol, and other preservatives conventionally used in pharmaceutical preparations, especially in creams. Suitable preservatives include methyl hydroxybenzoate, chlorocresol, sorbic acid and benzoic acid.

The compositions of the disclosure may be produced by conventional pharmaceutical techniques. Thus, ointments and creams are conveniently prepared by mixing together at an elevated temperature, optionally 60-70° C., the components constituting the vehicle until an emulsion has formed. The mixture may then be cooled to room temperature, and, after addition of the preferred enantiomer of a 2-hydroxycarboxylic acid, together with any other ingredients, stirred to ensure adequate dispersion.

Liquid preparations, such as ear and eye drops, are produced by dissolving the therapeutic agent in the components constituting the vehicle and the other ingredients are then added. The resulting solution or suspension is distributed into glass or plastic bottles or in single dose packs such as soft gelatine capsules which are then heat sealed.

Artificial tear vehicles may be used for ocular compositions comprising a preferred enantiomer of a 2-hydroxycarboxylic acid delivery. More viscous artificial tears use high concentrations of viscosity enhancing agents, such as Celluvisc®, high viscosity carboxymethyl cellulose (CMC) and Refresh Liquigel®, a blend of 0.35% high viscosity CMC and 0.65% low viscosity CMC.

Gelling agents may be used for compositions comprising a preferred enantiomer of a 2-hydroxycarboxylic acid being delivered to the eye. Such agents may be instilled as liquid and then almost immediately triggered to a gel phase. Timoptic gel (gellan gum), AzaSite® (polycarbophil, poloxamer), and Besivance®, (polycarbophil, poloxamer), 0.3% alginate Keltrol® are examples of such agents. Another gelling agent is polycarbophil-poloxamer gels (eg Durasite®).

The ocular carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminium monostearate and gelatine.

Nasal delivery of a preferred enantiomer of a 2-hydroxycarboxylic acid is also contemplated. Nasal delivery allows the passage of the preferred enantiomer of a 2-hydroxycarboxylic acid to the blood stream directly after administering the therapeutic product to the nose, without the necessity for deposition of the preferred enantiomer of a 2-hydroxycarboxylic acid in the lung. Compositions for nasal delivery include those with dextran or cyclodextran.

### Oral Delivery Excipients

The compositions of the present disclosure comprising a preferred enantiomer of a 2-hydroxycarboxylic acid may be delivered orally. For example, the oral formulations may be for local application to specific parts of the gastrointestinal system (such as the oesophagus, stomach or large or small intestines) or for a systemic application and may be delivered in a multitude of forms including a simple solution, syrup, suspension, tablet or capsule.

The compositions of the present disclosure may contain water (aqueous) or may be non-aqueous.

The composition of this disclosure may also include minor amounts of conventional additives such as preservatives, solubilizers (including ethanol), complexing agents (such as cyclodextrin), flowability enhancers and glidants (including colloidal silica), flavour enhancers and compactibility enhancers, including mixtures thereof. For some therapeutic agents it may be necessary to incorporate buffering agents to maintain a suitable pH.

The oral compositions of the present disclosure may be delivered with an immune boosting or modulating agents. Examples of immune boosting or modulating agents include specific immunostimulants (such as vaccines and antigens) and non-specific agents such as honeybee products (including propolis and honey), pro- and pre-biotics, hormones, vitamins (vitamin c, vitamin d), minerals (zinc oxide), antioxidants (including glutathione), interferons (including INF-alpha), interleukins (including interleukin 10), colchicine, thalidomide and imiquimod. The immune booster may be delivered at the same time as the preferred enantiomer of a 2-hydroxycarboxylic acid composition, or after the preferred enantiomer of a 2-hydroxycarboxylic acid composition.

In some situations, it is believed that administration of the preferred enantiomer of a 2-hydroxycarboxylic acid may sufficiently dysregulate the metabolism of the planktonic bacteria in the body such that concurrent or subsequent administration of the immune boosting agent allows the body to clear the planktonic bacteria without the use of further antimicrobial agents such as antibiotics.

### Plant Delivery Excipients

The composition may comprise active agrochemical compounds other than the preferred enantiomer of a 2-hydroxycarboxylic acid of the invention.

The composition may further comprise one or more of the following auxiliary components: inert carrier(s), surface active agent(s) such as a sticker or spreader, stabilizer(s) and/or dye(s). The compositions may also be suspended in a carrier fluid, such as air, nitrogen, carbon dioxide or fumigant gas. The compositions preferably comprise auxiliaries such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the composition, without the component itself having a biological effect. Examples of adjuvants are agents which promote the retention, spreading, attachment to the leaf surface, or penetration of the preferred enantiomer of a 2-hydroxycarboxylic acid into the plant tissue. Generally speaking, the active compounds may be combined with any solid or liquid additive commonly used for formulation purposes.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic or pseudoplastic thickeners), in-container microbial growth (antimicrobials), product freezing (antifreezes), colour (dyes/pigment dispersions), wash-off (film formers or sticking agents), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes.

### Stability

Optionally, the compositions of the present disclosure are stable. As used herein, the stability of compositions provided herein refers to the length of time at a given temperature that greater than 80%, 85%, 90% or 95% of the initial amount of antibiotic-lactic acid, is present in the composition. For example, the compositions provided herein may be stored between about 15° C. and about 30° C., and remain stable for at least 1, 2, 12, 18, 24 or 36 months. Also, the compositions may be suitable for administration to a subject in need thereof after storage for more than 1, 2, 12, 18, 24 or 36 months at 25° C. Also, in another alternative embodiment, using Arrhenius Kinetics, more than 80%, or more than 85%, or more than 90%, or more than 95% of the initial amount of active (e.g., the preferred enantiomer of a 2-hydroxycarboxylic acid) remains after storage of the compositions for more than 1, 2, 12, 18, 24 or 36 months between about 15° C. and about 30° C.

As used herein, the statement that a composition is stable during “long term storage” means that the composition is suitable for administration to a subject in need thereof when it has an estimated shelf-life of greater than 1, 2 or 3 months usage time at 25° C. and greater than or equal to 1, 2 or 3 years storage time at 5° C. In certain embodiments herein, using Arrhenius kinetics, an estimated >80% or >85% or >90% or >95% of the preferred enantiomer of a 2-hydroxycarboxylic acid remains after such storage.

### Antimicrobials

Other active agents may also be incorporated into the composition of the present disclosure. For example, additional antimicrobial compounds such as antibacterials, antifungals etc may be incorporated.

The additional active agent provided with the preferred enantiomer of a 2-hydroxycarboxylic acid of the present disclosure may be an active agent that kills bacteria, but is not an antibiotic. For example, the additional active agent may be a chlorine-based compound (such as bleach), an iodine-based compound, a copper-based compound, etc.

It is preferred that the preferred enantiomer of a 2-hydroxycarboxylic acid of the present disclosure dysregulates the metabolism of the planktonic bacteria such that the additional active agent (antibiotic or other compound capable of killing bacteria) is better able to have its bacterial killing effect. Optionally, the metabolism of the planktonic bacteria is dysregulated to such an extent that the bacteria cannot resist the effect of the additional active agent. It is further preferred that the preferred enantiomer of a 2-hydroxycarboxylic acid of the present disclosure dysregulates the metabolism of the planktonic bacteria such that the planktonic bacteria does not develop resistance to the additional active agent.

The dysregulation of the planktonic bacterial metabolism in the presence of the preferred enantiomer of a 2-hydroxycarboxylic acid alone may be suppression or enhancement of the planktonic bacterial metabolism. After introduction of the antimicrobial compound (so that the bacteria is exposed to both the antimicrobial compound and the 2-hydroxycarboxylic acid), the state of dysregulation (suppression or enhancement) may continue, perhaps with an increase in the degree of dysregulation. However, once the planktonic bacteria is in the presence of both the preferred enantiomer of a 2-hydroxycarboxylic acid and an antimicrobial compound, the dysregulation of the planktonic bacterial metabolism may alter, to be enhancement where there was once suppression, or suppression where there was once enhancement. Alternatively, there may be no dysregulation of metabolism in the presence of the 2-hydroxycarboxylic acid alone, but significant dysregulation in the presence of the preferred enantiomer of a 2-hydroxycarboxylic acid and an antimicrobial compound, particularly where the planktonic bacterium was resistant to the antimicrobial compound in the absence of the preferred enantiomer of a 2-hydroxycarboxylic acid.

The composition may further comprise benzoyl peroxide, or an antibiotic such as erythromycin, clindamycin, doxycycline or meclocycline.

Additional antimicrobial compounds that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Use

The present disclosure provides for the use of a preferred enantiomer of a 2-hydroxycarboxylic acid in the manufacture of a composition for the dysregulation of planktonic bacterial metabolism.

The present disclosure further provides for the use of a preferred enantiomer of a 2-hydroxycarboxylic acid for the dysregulation of planktonic bacterial metabolism.

The present disclosure provides for the use of a preferred enantiomer of a 2-hydroxycarboxylic acid in the manufacture of a composition for the dysregulation of planktonic bacterial metabolism in combination with an antimicrobial compound.

The present disclosure further provides for the use of a preferred enantiomer of a 2-hydroxycarboxylic acid for the dysregulation of planktonic bacterial metabolism in combination with an antimicrobial compound.

The present disclosure further provides for the use of a preferred enantiomer of a 2-hydroxycarboxylic acid in the manufacture of a composition for treating or preventing an infection in a subject wherein the infection is by planktonic bacteria, and wherein the composition dysregulates the metabolism of the planktonic bacterium.

The present disclosure further provides for the use of a preferred enantiomer of a 2-hydroxycarboxylic acid in the manufacture of a composition for treating or preventing an infection in a subject, in combination with an antimicrobial compound, wherein the infection is by planktonic bacteria, and wherein the composition dysregulates the metabolism of the planktonic bacterium.

The present disclosure further provides for the use of a preferred enantiomer of a 2-hydroxycarboxylic acid for treating or preventing an infection in a subject wherein the infection is by planktonic bacteria, and wherein the composition dysregulates the metabolism of the planktonic bacterium.

The present disclosure further provides for the use of a preferred enantiomer of a 2-hydroxycarboxylic acid for treating or preventing an infection in a subject, in combination with an antimicrobial compound wherein the infection is by planktonic bacteria, and wherein the composition dysregulates the metabolism of the planktonic bacterium.

The present disclosure provides for the use of a preferred enantiomer of a 2-hydroxycarboxylic acid in the manufacture of a composition for sensitising planktonic bacteria to an antimicrobial compound, wherein the preferred enantiomer of a 2-hydroxycarboxylic acid induces dysregulation of the planktonic bacterial metabolism.

The present disclosure further provides for the use of a preferred enantiomer of a 2-hydroxycarboxylic acid for sensitising planktonic bacteria to an antimicrobial compound, wherein the preferred enantiomer of a 2-hydroxycarboxylic acid induces dysregulation of the planktonic bacterial metabolism.

The present disclosure provides for the use of a preferred enantiomer of a 2-hydroxycarboxylic acid in the manufacture of a composition for sensitising planktonic bacteria to an antimicrobial compound, wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

The present disclosure further provides for the use of a preferred enantiomer of a 2-hydroxycarboxylic acid for sensitising planktonic bacteria to an antimicrobial compound, wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

The present disclosure further provides for the use of a preferred enantiomer of a 2-hydroxycarboxylic acid in the manufacture of a composition for treating or preventing an infection in a subject, wherein the infection is by planktonic bacteria, wherein the composition sensitises the planktonic bacterium to an antimicrobial compound, and wherein the preferred enantiomer of a 2-hydroxycarboxylic acid induces dysregulation of the planktonic bacterial metabolism.

The present disclosure further provides for the use of a preferred enantiomer of a 2-hydroxycarboxylic acid in the manufacture of a composition for treating or preventing an infection in a subject, in combination with an antimicrobial compound, wherein the infection is by planktonic bacteria, wherein the composition sensitises the planktonic bacterium to the antimicrobial compound, and wherein the preferred enantiomer of a 2-hydroxycarboxylic acid induces dysregulation of the planktonic bacterial metabolism.

The present disclosure further provides for the use of a preferred enantiomer of a 2-hydroxycarboxylic acid for treating or preventing an infection in a subject, wherein the infection is by planktonic bacteria, wherein the composition sensitises the planktonic bacterium to an antimicrobial compound, and wherein the preferred enantiomer of a 2-hydroxycarboxylic acid induces dysregulation of the metabolism of the planktonic bacterium.

The present disclosure further provides for the use of a preferred enantiomer of a 2-hydroxycarboxylic acid for treating or preventing an infection in a subject, in combination with an antimicrobial compound, wherein the infection is by planktonic bacteria, wherein the composition sensitises the planktonic bacterium to the antimicrobial compound, and wherein the preferred enantiomer of a 2-hydroxycarboxylic acid induces dysregulation of the planktonic bacterial metabolism.

In the uses above, the dysregulation of the planktonic bacterial metabolism in the presence of the preferred enantiomer of a 2-hydroxycarboxylic acid alone may be suppression or enhancement of the planktonic bacterial metabolism. After introduction of the antimicrobial compound, the state of dysregulation (suppression or enhancement) may continue, perhaps with an increase in the degree of dysregulation. However, once the planktonic bacteria is in the presence of both the preferred enantiomer of a 2-hydroxycarboxylic acid and an antimicrobial compound, the dysregulation of the planktonic bacterial metabolism may alter, to be enhancement where there was once suppression, or suppression where there was once enhancement. Alternatively, there may be no dysregulation of metabolism in the presence of the 2-hydroxycarboxylic acid alone, but significant dysregulation in the presence of the preferred enantiomer of a 2-hydroxycarboxylic acid and an antimicrobial compound, particularly where the planktonic bacterium was resistant to the antimicrobial compound in the absence of the preferred enantiomer of a 2-hydroxycarboxylic acid.

The preferred enantiomer of a 2-hydroxycarboxylic acid may be in the same composition as the antimicrobial compound, or may be in separate compositions. The planktonic bacteria may be associated with or cause an infection of a subject, or the planktonic bacteria may be present on a surface, such as a non-living surface. The planktonic bacteria may be present in a liquid (living or non-living) or gas.

If the preferred enantiomer of a 2-hydroxycarboxylic acid is in a separate composition to the antimicrobial compound, the preferred enantiomer of a 2-hydroxycarboxylic acid may be administered at the same time as the antimicrobial compound, or may be administered at a different time as the antimicrobial compound.

For the above uses, the preferred enantiomer of a 2-hydroxycarboxylic acid may be D-lactic acid. Optionally, the infection or colonisation is infection or colonisation by a bacteria.

### Kits

The present disclosure provides a kit for dysregulation of the metabolism of planktonic bacteria comprising:
a) a preferred enantiomer of a 2-hydroxycarboxylic acid; and
b) instructions for use.

The present disclosure provides a kit for dysregulation of the metabolism of planktonic bacteria comprising:
a) a preferred enantiomer of a 2-hydroxycarboxylic acid; and
b) instructions for use
   wherein the preferred enantiomer of a 2-hydroxycarboxylic acid is in combination with an antimicrobial compound.

The present disclosure provides a kit for sensitising planktonic bacteria to an antimicrobial compound, comprising:
a) a preferred enantiomer of a 2-hydroxycarboxylic acid; and
b) instructions for use.
   wherein the preferred enantiomer of a 2-hydroxycarboxylic acid induces dysregulation of the planktonic bacterial metabolism.

The present disclosure provides a kit for sensitising planktonic bacteria to an antimicrobial compound, comprising:
a) a preferred enantiomer of a 2-hydroxycarboxylic acid; and
b) instructions for use
   wherein the preferred enantiomer of a 2-hydroxycarboxylic acid induces dysregulation of the planktonic bacterial metabolism, and wherein the preferred enantiomer of a 2-hydroxycarboxylic acid is in combination with the antimicrobial compound.

In the kits above, the dysregulation of the planktonic bacterial metabolism in the presence of the preferred enantiomer of a 2-hydroxycarboxylic acid alone may be suppression or enhancement of the planktonic bacterial metabolism. After introduction of the antimicrobial compound, the state of dysregulation (suppression or enhancement) may continue, perhaps with an increase in the degree of dysregulation. However, once the planktonic bacteria is in the presence of both the preferred enantiomer of a 2-hydroxycarboxylic acid and an antimicrobial compound, the dysregulation of the planktonic bacterial metabolism may alter, to be enhancement where there was once suppression, or suppression where there was once enhancement. Alternatively, there may be no dysregulation of metabolism in the presence of the 2-hydroxycarboxylic acid alone, but significant dysregulation in the presence of the preferred enantiomer of a 2-hydroxycarboxylic acid and an antimicrobial compound, particularly where the planktonic bacterium was resistant to the antimicrobial compound in the absence of the preferred enantiomer of a 2-hydroxycarboxylic acid.

The preferred enantiomer of a 2-hydroxycarboxylic acid of the kit may be D-lactic acid.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the composition may be applied to an affected area of the animal, such as the lungs, injected into an animal, or even applied to and mixed with the other components of the kit.

In an embodiment, the kit of the present disclosure comprises a composition comprising a preferred enantiomer of a 2-hydroxycarboxylic acid, optionally in combination with an antimicrobial compound. In an alternative embodiment, the composition is in pre-measured, pre-mixed and/or pre-packaged.

The kit of the present disclosure may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present disclosure comprises instructions for administering the compositions of the present disclosure. In one embodiment, the instructions indicate that the composition of the present disclosure is suitable for the dysregulation of planktonic bacterial metabolism. Such instructions may also include instructions on dosage, as well as instructions for administration.

The preferred enantiomer of a 2-hydroxycarboxylic acid and suitable excipients can be packaged individually so to allow a practitioner or user to formulate the components into a pharmaceutically acceptable composition as needed. Alternatively, the antisense oligomers and suitable excipients can be packaged together, thereby requiring de minimis composition by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, compositions and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Suppression of Planktonic Bacteria Metabolism

This study demonstrates that D-Lactic acid suppresses the metabolism of planktonic bacteria.

### Materials and Methods

### Planktonic Bacterial Culture

Clinical isolates of Pseudomonas aeruginosa collected from patients with cystic fibrosis were phenotyped and stored as 50% glycerol stocks at −70° C.±2° C. Clinical isolates were thawed and streaked out on blood agar plates to grow at 37° C., 5% CO₂ for 12-24 hours, or until isolated colonies were large enough to sustain further culturing could be selected. Isolated colonies were looped into 5 mL of LB broth and incubated overnight at 37° C., 5% CO₂ with gentle shaking to achieve stationary phase (B₀).

B₀ cultures were diluted 1:1000 in Pseudomonas minimal medium (herein referred to as PMM) to achieve approximately 10⁸ cfu/mL, then 150 μL of each diluted culture was transferred to wells of a 96 well mitrotitre tray and incubated overnight at 37° C., 5% CO₂. D-Lactic acid was then applied by adding 50 μL of appropriately stock solutions diluted in PMM (or vehicle-only in the case of control wells) and incubating for a further 24 hours at 37° C., 5% CO₂. 20 μL culture supernatant was then transferred to wells containing 180 μL fresh PMM, culture density determined by measuring the absorbance at 595 nm (using a ThermoFisher Multiskan FC plate reader). These diluted cultures were then used to determine metabolic activity with resazurin staining.

### Resazurin Staining

Resazurin stain was used to measure changes in bacterial metabolic activity following treatment (Kirchner et al 2012). 10 μl of 0.02% (v/v) resazurin (diluted in distilled water) was added to each well and the microtite plates incubated under aerobic conditions for 1-2 h at 37° C., while shaking at 150 rpm. Viable cells reduce the blue resazurin dye to the pink, fluorescent resorufin form. Following incubation fluorescence was quantified by excitation wavelength of 540 nm and an emission wavelength of 590 nm using Enspire microplate reader.

### Statistical Analysis

All data presented represents the data from 24 clinical isolates of Pseudomonas aeruginosa containing 2 technical duplicates unless otherwise stated. Students t-test used to assess significance compared to control untreated biofilms with significance measured at p<0.05.

### Results

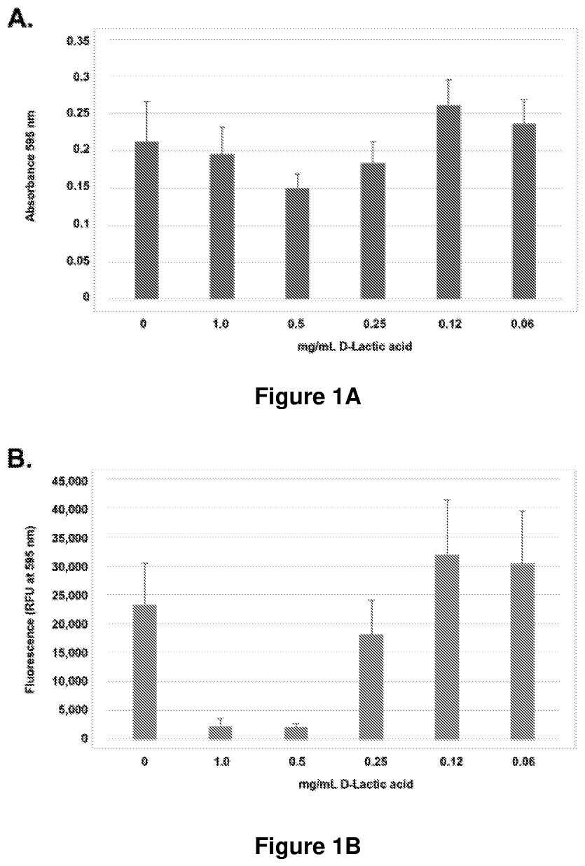

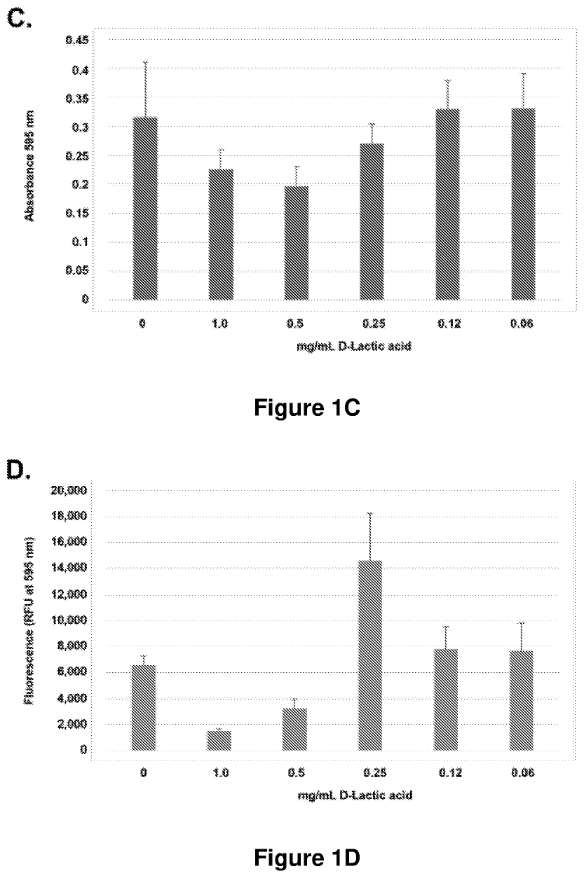

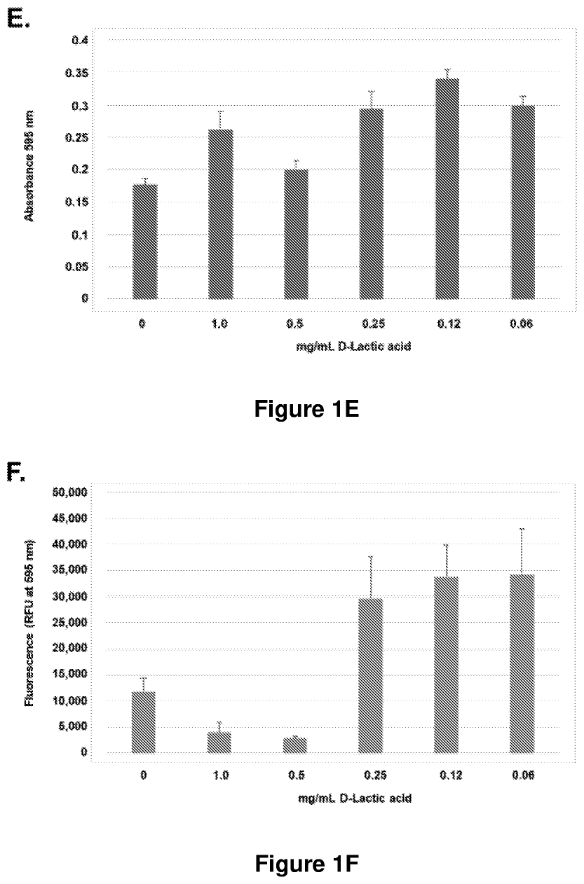

Resorufin fluorescence was measured to quantify the impact of D-Lactic acid on the metabolic activity of planktonic cultures. 90% inhibition of metabolic activity was observed for cultures treated with D-Lactic acid, with the change of activity being clearest between 0.25 mg/mL and 0.5 mg/mL D-Lactic acid (FIG. 1).

D-Lactic acid does not significantly reduce P. aeruginosa planktonic biomass but does have a significant impact on baseline metabolic activity.

### Example 2

### Suppression of Planktonic Bacteria Metabolism Increases Microbial Susceptibility to Antimicrobial Compounds

This study demonstrates that D-Lactic acid treatment increases the susceptibility of planktonic bacteria to antibiotics, to the extent that antimicrobial resistance is reversible with D-Lactic acid treatment.

### Materials and Methods

### Planktonic Bacterial Culture

Bacterial strains were maintained as suspensions in 20% (v/v) glycerol/Brain-Heart Infusion at −80° C. For use, a loop-full of ice was scraped from the stock and streaked for single colonies on rich media containing 1.8% agar (for Pseudomonas aeruginosa or Klebsiella pneumoniae, Luria-Bertani medium; for Staphylococcus aureus or Streptococcus pneumoniae, Trypticase Soy agar) and incubated overnight at 37° C.

### Minimum Inhibitory Concentrations by Broth Microdilution

A standard inoculum was prepared from broth culture. 1 to 3 colonies from an overnight agar culture plate were transferred to 5 ml of broth media and incubated at 37° C. with shaking (200 rpm) until turbidity was greater than that of a 0.5 McFarland Standard (overnight). The following broth media were used: Cation-adjusted Mueller-Hinton broth (caMHB—all strains); Roswell Park Memorial Institute medium (RPMI 1640, phenol red-free—S. aureus); Trypticase Soya Broth (TSB—S. aureus, S. pneumoniae); Todd Hewitt Broth (THB—S. pneumoniae), M63 minimal medium, supplemented with 0.2% glucose (M63—K. pneumoniae).

The broth culture was diluted in broth to a turbidity equal to a 0.5 McFarland Standard using a spectrophotometer at a wavelength of 620 nm. The 0.5 McFarland Standard was further diluted 1:100 in broth to create the standard inoculum. 50 μl of this standard inoculum was dispensed into every well in the microtitre tray, except the negative controls, where 50 μl of broth was dispensed instead.

Next, a working concentration at two times the highest target concentration for every antimicrobial was created in broth from stock solutions. 100 μl of this working concentration was pipetted into the first well, and 50 μl of broth media into all other wells. 50 μl of the first well was pipetted out and serially diluted across all wells in the row until the final concentration in the range achieved. 50 μl from the final well was removed and discarded. This produced a concentration range where each well was double the target concentration. All agents were performed in duplicate.

96 well trays were incubated at 37° C. for 18±2 hours. Following incubation, MICs were evaluated visually and recorded as the lowest concentration of antimicrobial that completely inhibited bacterial growth determined as a complete lack of turbidity.

### Resazurin Reduction for the Evaluation of Metabolic Activity

Aerobic respiration was measured in the presence or absence of D-Lactic acid and the presence or absence of companion antibiotics either following incubation and visual recording of the MIC, or in a separate study. For evaluation after completion of MIC determination, the entire volume of the MIC well (100 μl) was mixed by pipetting up and down, then transferred to the equivalent well of a fluorescence-absorbance suitable 96-well plate. Alternatively, cultures were pipetted together as above and incubated overnight at 37° C. in the absence of any antimicrobial compounds. Culture supernatants were removed, adherent cells washed and then treated by the addition of 100 μL of fresh broth, 50 μL of broth containing 4×the desired concentration of antibiotic and 50 μL containing 4×the desired concentration of D-Lactic acid or vehicle only controls, followed by incubation at 37° C. for a further 18±2 hours. After incubation regimes were completed for both procedures, resazurin reduction was evaluated by adding 10 μl of a 0.02% resazurin solution to every well, and incubating the plates with gentle shaking (40 rpm) for a further 90 minutes at 37° C. 96-well plates and resazurin were protected from light as much as possible.

Following incubation, the 96-well trays were read on a fluorescence-absorbance spectrophotometer, using an excitation frequency of 565 nm and emission frequency of 590 nm. The value for each well was blanked by subtracting the value of the negative control. Every value was then converted to a ratio with respect to the mean value of the positive controls.

### Results

### Minimal Inhibitory Concentration (MIC) is Reduced by Co-Administration of 0-Lactic Acid for Multiple Antibiotics.

First, the MICs for multiple antibiotics, either alone or in combination with 0-Lactic acid, were determined for medically relevant strain/antibiotic combinations. The results are shown in Table 3 and indicate that 0-Lactic acid treatment leads to a minimum two-fold reduction in the MIC, i.e. a minimum two-fold enhancement of the potency of the antibiotic for the bacterial strain in question.

TABLE 3
Effect on antibiotic MIC of co-administration of D-Lactic acid

| | | | MIC + 1 mM | MIC + 10 mM |
|---|---|---|---|---|
| Strain | Antibiotic | MIC | D-Lactic acid | D-Lactic acid |
| P. aeruginosa | aztreonam | 8.0 | 4.0 | 4.0 |
| WACC91 | meropenem | 1.0 | <0.5 | <0.5 |
| P. aeruginosa | colistin | 2.0 | 1.0 | 1.0 |
| 001-11R | | | | |
| S. aureus 29213 | amoxicillin | 4.0 | <1.0 | 2.0 |
| (MSSA) | ceftriaxone | 8.0 | 4.0 | 4.0 |
| | meropenem | 1.0 | 0.5 | 0.5 |
| | methicillin | 1.0 | 0.5 | 0.5 |
| | vancomycin | 8.0 | 4.0 | 2.0 |
| S. aureus | rifampicin | 0.015 | 0.008 | 0.008 |
| MSSA 278 | | | | |
| S. aureus 510 | ceftriaxone | 16.0 | 8.0 | 8.0 |
| (MRSA) | meropenem | >4.0 | 2.0 | 2.0 |
| | methicillin | >16.0 | 2.0 | 2.0 |
| | rifampicin | 0.06 | 0.008 | 0.008 |
| | tetracycline | 1.0 | <0.5 | <0.5 |
| | vancomycin | 8.0 | 2.0 | 2.0 |
| S. pneumoniae | amoxicillin | 0.5 | 0.25 | 0.25 |
| D39 | ampicillin | >16.0 | 4.0 | 4.0 |
| | meropenem | 0.5 | 0.25 | 0.5 |

### Suppression of Planktonic Bacterial Metabolism by 0-Lactic Acid is Further Enhanced by Antibiotic Treatment.

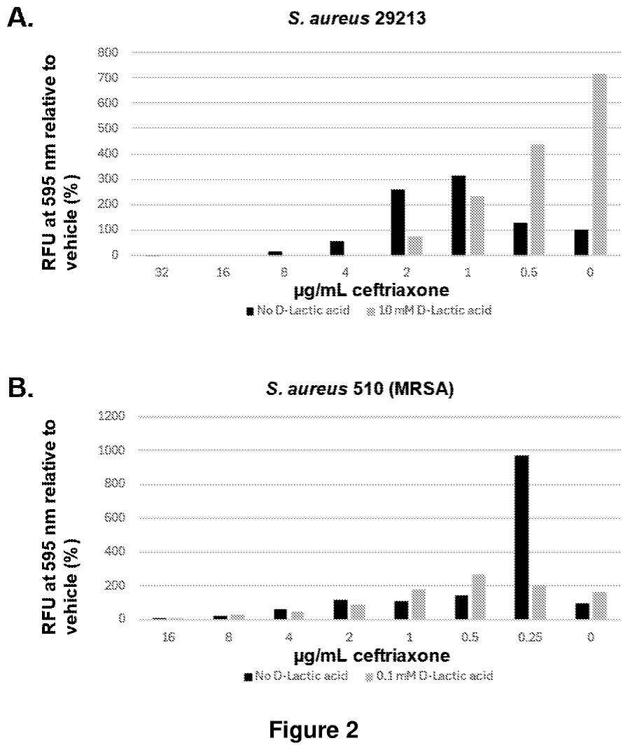

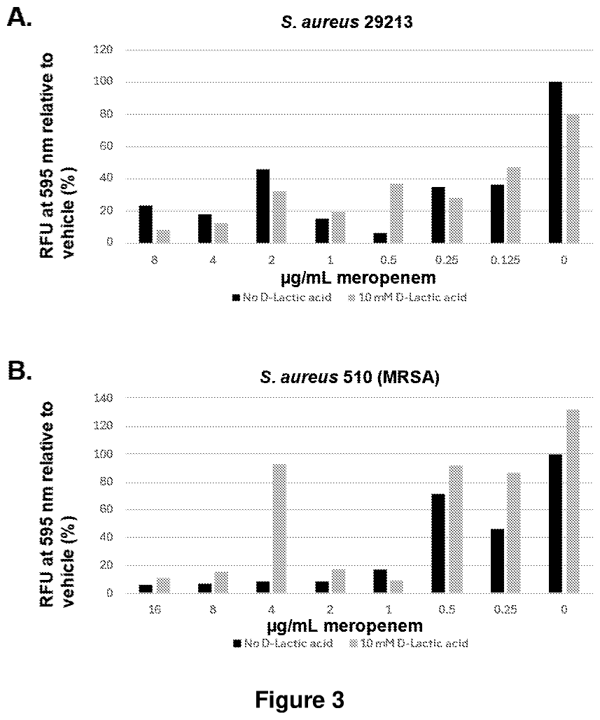

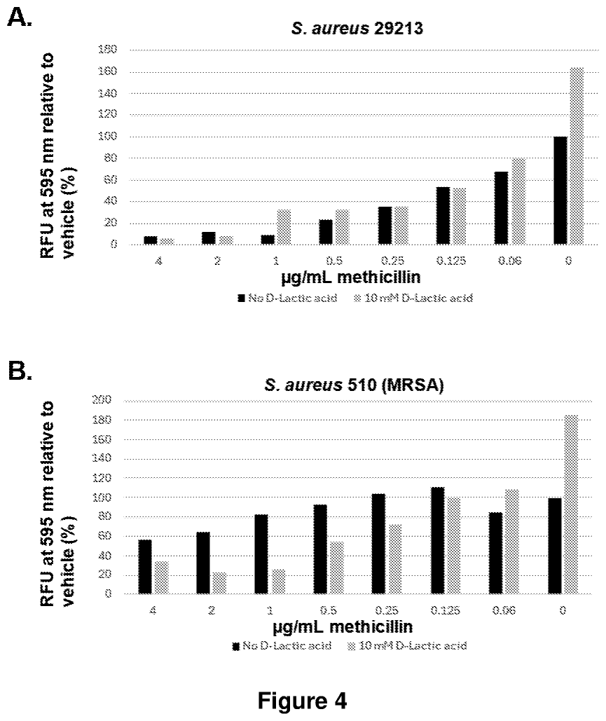

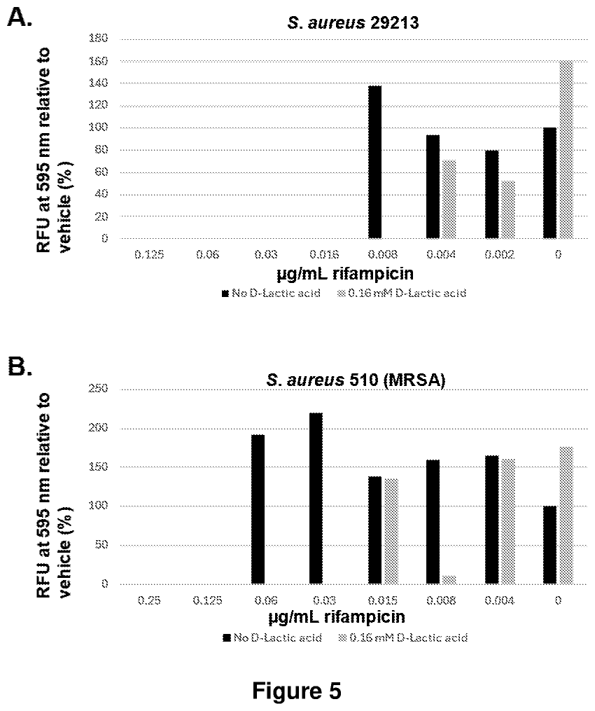

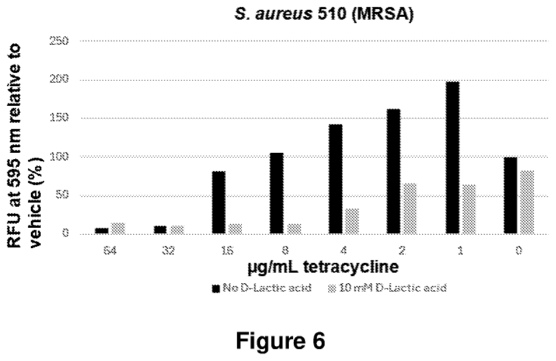

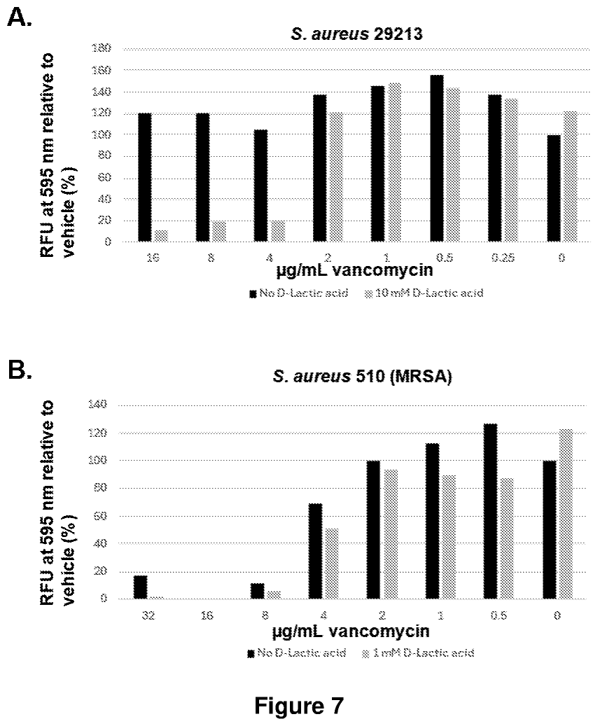

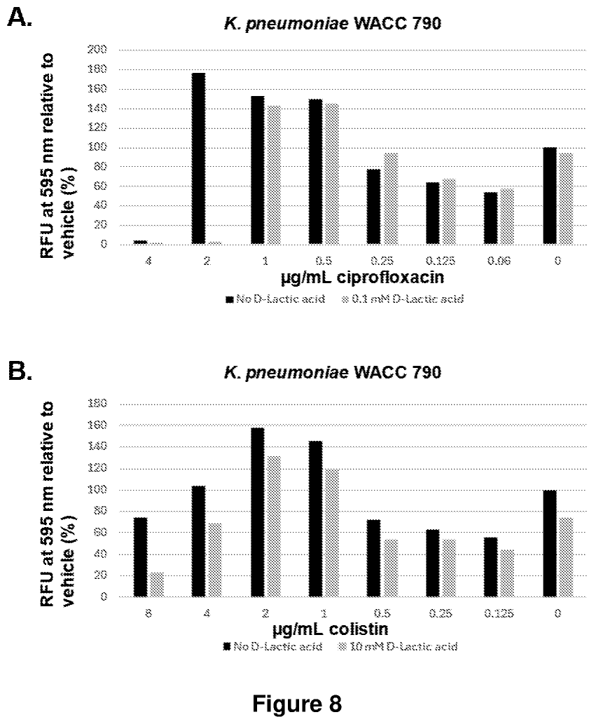

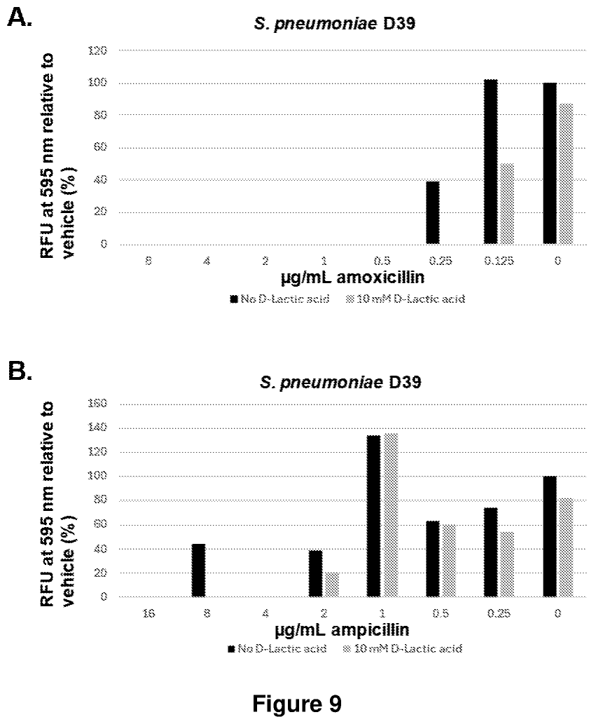

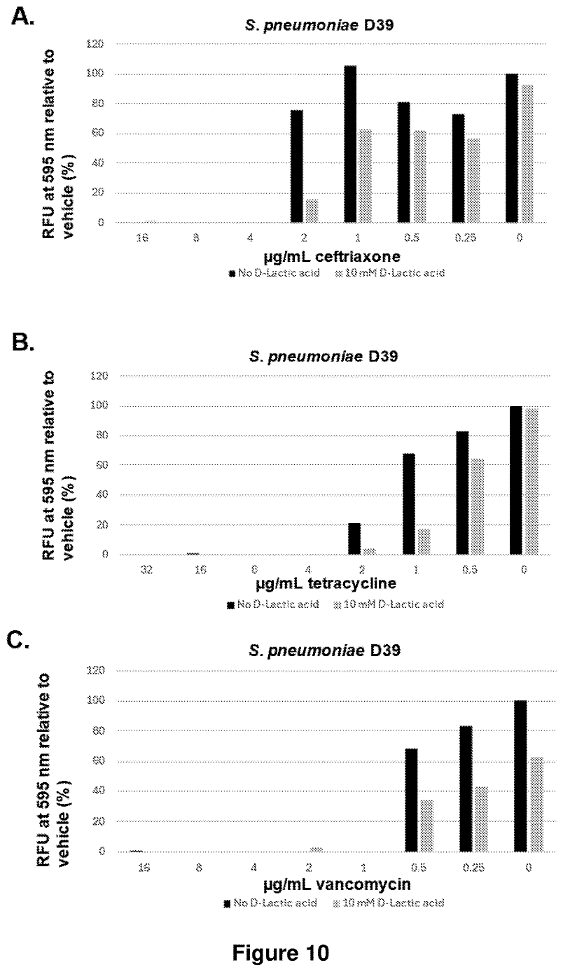

Resorufin fluorescence was measured to quantify the impact of 0-Lactic acid on the metabolic activity of planktonic cultures when combined with either bactericidal or bacteriostatic antibiotics. The data is summarised in Table 4 and shown in FIGS. 2-10.

TABLE 4
Effect of D-Lactic acid administration on bacterial
metabolic activity with or without a
companion antimicrobial, as measured by some of the metabolic
dysregulation indicators, such as resazurin activity
fluctuations, or a reduction in companion antimicrobial MIC

| | | | | Antimicrobial |
|---|---|---|---|---|
| | | Resazurin | Resazurin | efficacy (MIC) |
| | | activity (D- | activity (D- | enhancement |
| | | Lactic acid | Lactic acid plus | with |
| Strain | Antibiotic | only)¹ | antimicrobial)² | D-Lactic acid³ |
| P. aeruginosa WACC91 | aztreonam | ↓ | NT | ↑ |
| | meropenem | ↓ | NT | ↑ |
| P. aeruginosa 001-11R | colistin | NT | NT | ↑ |
| S. aureus 29213 (MSSA) | amoxicillin | ↑ | NT | ↑ |
| | ceftriaxone | ↑ | ↓ | ↑ |
| | meropenem | ↑ | ↑ | ↑ |
| | methicillin | ↑ | ↑ | ↑ |
| | vancomycin | ↑ | ↓ | ↑ |
| S. aureus MSSA 278 | rifampicin | ↑ | ↓ | ↑ |
| S. aureus 510 (MRSA) | ceftriaxone | ↑ | NT | ↑ |
| | meropenem | ↑ | ↑ | ↑ |
| | methicillin | ↑ | ↓ | ↑ |
| | rifampicin | ↑ | ↓ | ↑ |
| | tetracycline | ↑ | ↓ | ↑ |
| | vancomycin | ↑ | ↓ | ↑ |
| S. pneumoniae D39 | amoxicillin | ↓ | ↓ | ↑ |
| | ampicillin | ↓ | ↓ | ↑ |
| | ceftriaxone | NT | ↓ | NT |
| | meropenem | ↓ | | ↑ |
| | tetracycline | ↓ | ↓ | NT |
| | vancomycin | ↓ | ↓ | NT |

¹Resazurin activity refers to an increase (↑) or decrease (↓) in resazurin reduction when treated with D-Lactic acid in the absence of any companion antimicrobial compared to vehicle only treatment.
²Resazurin activity refers to an increase (↑) or decrease (↓) in resazurin reduction when treated with a combination of D-Lactic acid and the indicated companion antimicrobial in comparison with vehicle only treatment.
³Antimicrobial efficacy enhancement refers to an increase (↑) or decrease (↓) in the efficacy of the indicated antimicrobial (ie a decrease or increase in its MIC, respectively) if D-Lactic acid is present compared to the efficacy (MIC measured in vitro) in its absence.
In all cases, “NT” refers to “Not tested”.

The observed reduction in metabolic activity upon antibiotic treatment was further enhanced in combination with 0-Lactic acid. In each of FIGS. 2-10, the histograms show the amount of metabolic activity measured by resorufin fluorescence relative to vehicle-only controls. Relative metabolic activity is indicated in black for antibiotic-only treatments and in grey for antibiotic plus D-Lactic acid treatments.

### Example 3

### Dysregulation of Planktonic Bacteria Metabolism is Specific to the D-Enantiomer

This study demonstrates that the dysregulation of metabolism observed for P. aeruginosa in the presence of D-Lactic acid is not replicated in the presence of L-Lactic acid.

### Materials and Methods

### Planktonic Bacterial Culture

P. aeruginosa strain WACC91 was maintained as a suspension in 20% (v/v) glycerol/Brain-Heart Infusion at −80° C. For use, a loop-full of ice was scraped from the stock and streaked for single colonies on LB media containing 1.8% agar and incubated overnight at 37° C. Isolated colonies were looped into 5 mL of LB broth and incubated at 37° C. with shaking until turbidity was greater than that of a 1.0 McFarland Standard (3-4 h).

The broth culture was diluted in M63 minimal media containing 0.2% (w/v) glucose as the sole carbon source to a turbidity equal to a 1.0 McFarland standard using a spectrophotometer at a wavelength of 595 nm. This culture was then diluted 1:100 in M63 (glucose) and 100 μL together with an additional 50 μL was transferred to each well of a 96 well microtitre tray and incubated overnight at 37° C., 5% CO₂. D-Lactic acid, L-Lactic acid, or vehicle only (sterile H2O) was then applied by adding 50 μL of appropriate stock solutions diluted in M63 (glucose) and incubating for a further 24 hours at 37° C., 5% CO₂. Culture metabolic activity was then determined using resazurin reduction as described above in Example 1.

### Results

Resorufin fluorescence was measured to quantify the impact of D-Lactic acid or L-Lactic acid on the metabolic activity of planktonic cultures. >50% inhibition of metabolic activity was observed for cultures treated with D-Lactic acid (compared to vehicle only treatment), with the change of activity being clearest between 12.5 mM and 25 mM D-Lactic acid (FIG. 1). No change in metabolic activity was observed for cultures treated with L-Lactic acid. Therefore, the metabolic dysregulation observed is specific for the D-enantiomer of Lactic acid.

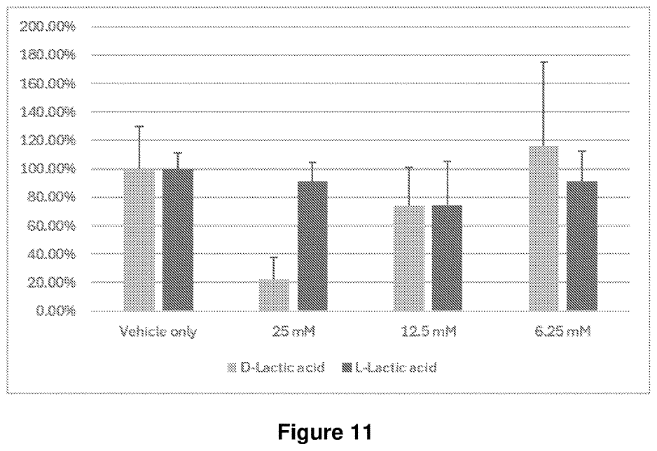

FIG. 11 is a histogram of the outcome of adding 2-fold decreasing concentrations of D-Lactic acid or L-Lactic acid to planktonic cultures of P. aeruginosa WACC91. The figure shows that treatment range results in a breakpoint between 12.5 mM and 25 mM D-Lactic acid when metabolic rate (Resazurin reduction to the fluorescent Resorufin) in comparison to vehicle-only treatment is used as a readout. No comparable breakpoint is observed when L-Lactic acid is added at the same concentrations as the effect is observed for D-Lactic acid.

### Example 4

### D-Lactate Salts Enhance the Antibacterial Activity of Metal Ions

This study demonstrates that the antibacterial activity of metal ions against planktonic bacteria is enhanced if the metal ion is present as a D-Lactate salt.

### Materials and Methods

### Planktonic Bacterial Culture

Bacterial strains were maintained as suspensions in 20% (v/v) glycerol/Brain-Heart Infusion at −80° C. For use, a loop-full of ice was scraped from the stock and streaked for single colonies on Trypticase Soya agar containing 1.8% agar and incubated overnight at 37° C.

### Minimum Inhibitory Concentrations by Broth Microdilution

A standard inoculum was prepared from broth culture. One to three colonies from an overnight agar culture plate were transferred to 5 ml of broth media and incubated at 37° C. with shaking (200 rpm) until turbidity was greater than that of a 0.5 McFarland Standard (overnight). All assays were performed in Trypticase Soya broth.

The broth culture was diluted in broth to a turbidity equal to a 0.5 McFarland Standard using a spectrophotometer at a wavelength of 620 nm. The 0.5 McFarland Standard was further diluted 1:100 in broth to create the standard inoculum. 50 μl of this standard inoculum was dispensed into every well in the microtitre tray, except the negative controls, where 50 μl of broth was dispensed instead.

Next, a working concentration at two times the highest target concentration for every salt (in aqueous solution) was created in broth from stock solutions. 100 μl of this working concentration was pipetted into the first well, and 50 μl of broth media into all other wells. 50 μl of the first well was pipetted out and serially diluted across all wells in the row until the final concentration in the range achieved. 50 μl from the final well was removed and discarded. This produced a concentration range where each well was double the target concentration. All agents were performed in duplicate.

96 well trays were incubated at 37° C. for 18±2 hours. Following incubation, MICs were evaluated visually and recorded as the lowest concentration of antimicrobial that completely inhibited bacterial growth determined as a complete lack of turbidity.

### Results

### Minimal Inhibitory Concentration (MIC) is Reduced in the Presence of D-Lactate

MICs for Zinc (Zn) or Copper (Cu) salts, present as either nitrate or D-Lactate salts, were determined. The results are shown in Table 4 and indicate the presence of D-Lactate leads to a minimum of a two-fold reduction in the MIC, that is, a minimum two-fold enhancement of the potency of the metal ion for the bacterial strain in question.

TABLE 5
Effect on metal ion MIC of administration as a Nitrate or D-Lactate salt

| | | MIC | MIC |
|---|---|---|---|
| Metal ion | Bacterial strain | (NO₃)₂ salt | D-Lactate salt |
| Zn²⁺ | S. aureus 29213 | 8 mM | 4 mM |
| | S. aureus MRSA 510 | 8 mM | 4 mM |
| Cu²⁺ | S. aureus MRSA 510 | 8 mM | 4 mM |

### Example 6

### Suppression of Planktonic Bacteria Metabolism Increases Microbial Susceptibility to Antimicrobial Agents

This study demonstrates that D-Lactic acid treatment increases the susceptibility of a colistin resistant strain of Escherichia coli to colistin in a murine peritoneal-sepsis model, to the extent that when comparing treatment with D-Lactic acid combined with colistin to treatment with colistin alone: (i) disease severity is mitigated as measured by viable counts of bacteria in blood samples and imaging of bacterial load in live animals; and (ii) survival of the mice is increased.

### Materials and Methods

### Bacterial Inoculum Preparation

Bioluminescent col^{R} E. coli Xen14 was streaked onto a 5% sheep blood agar plate containing 30 μg/ml kanamycin and incubated overnight (o/n) at 37° C. in normal air. The plate was first checked on a Lumina XRMS Ill machine to ensure bioluminescence, then a loopful of the o/n growth was suspended into 10 mL Luria Bertani (LB) broth to A₆₀₀ nm=0.05. The suspension was incubated at 37° C. for approx. 3 h until A₆₀₀ nm=0.5 (equivalent to approx. 5×10⁸ CFU/mL). The bacterial suspension was centrifuged at 4,000×g for 5 min, resuspended and washed twice in phosphate buffered saline (PBS, pH 7.2) resuspended again in PBS and then placed on wet ice and used for mouse infection within 10 min of preparation as described below.

### Mouse Infection Studies

Outbred Swiss male (CD1) mice, 6-7 weeks (approx. 26-33 g, 8 mice per group) obtained from the Laboratory Animal Services breeding facility of The University of Adelaide, were used in these experiments. Mice had access to food and water ad libitum. The Animal Ethics Committee of The University of Adelaide (approval number S-2022-044) reviewed and approved all animal experiments. The study was conducted in compliance with the Australian Code of Practice for the Care and Use of Animals for Scientific Purposes (8th Edition 2013) and the South Australian Animal Welfare Act 1985.

Each mouse was challenged intraperitoneally (IP) with approx. 1×10⁸ CFU of the bioluminescent col^{R} E. coli Xen14 in 200 μL PBS containing 3% porcine stomach mucin type Ill (Sigma Aldrich) and subjected to bioluminescence imaging in both ventral and dorsal positions on the IVIS Lumina XRMS Series Ill system while under anaesthesia. Immediately after, efficacy testing of Sodium D-Lactate (1000 mg/kg; pH 7.03) was performed as follows:

### Groups:

Group 1: A single oral dose of vehicle-only control (sterile water only)
Group 2: A single oral dose of 1000 mg/kg of Sodium D-Lactate (active 1) in water
Group 3: A single oral dose of 1000 mg/kg of Sodium D-Lactate at 0 h+4 IP doses of 1
mg/kg colistin (col) at 0, 4, 8 and 12 h (active 2)
Group 4: 4 IP doses of 1 mg/kg colistin (col) at 0, 4, 8 and 12 h (sub-therapeutic control drug)

Aliquots of blood samples (approx. 50 μL) were taken by submandibular (facial vein) bleeding from individual mice in all groups at 4 h for bacterial enumeration. Mice were monitored frequently for signs of distress up till 72 h post-infection, their clinical conditions recorded, imaged and those that had become moribund or showed any evidence of distress were humanely killed by CO₂ asphyxiation. In all experiments, signals were collected from a defined region of interest and total flux intensities (photons/s) analysed using Living Image Software 4.7.3. Survival rates for the groups were recorded. Differences in bioluminescence signals between groups were compared by unpaired t-tests (two-tailed).

### Results

As detailed above, mice were challenged intraperitoneally (IP) with approx. 1×10⁸ CFU of the bioluminescent col^{R} E. coli Xen14 in 200 μL PBS and then treated with either a single oral dose of sterile water (negative control), a single oral dose of Sodium D-Lactate (1000 mg/kg), a single oral dose of Sodium D-Lactate (1000 mg/kg) combined with four IP doses of colistin (1 mg/kg at 0, 4, 8 and 12 h), or four IP doses of colistin (1 mg/kg at 0, 4, 8 and 12 h) alone.

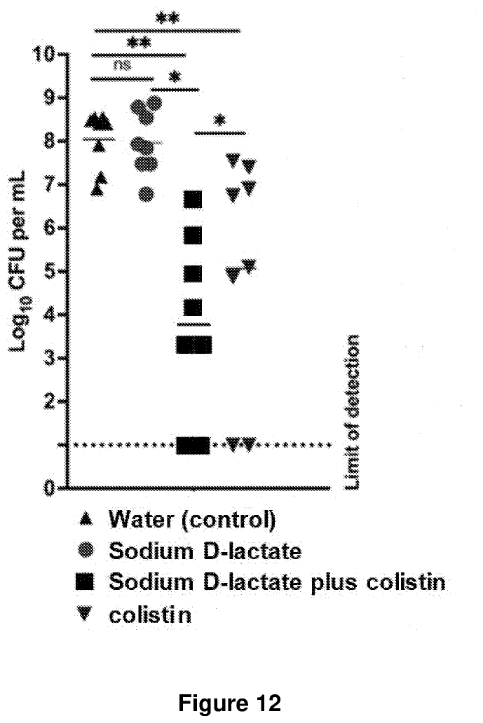

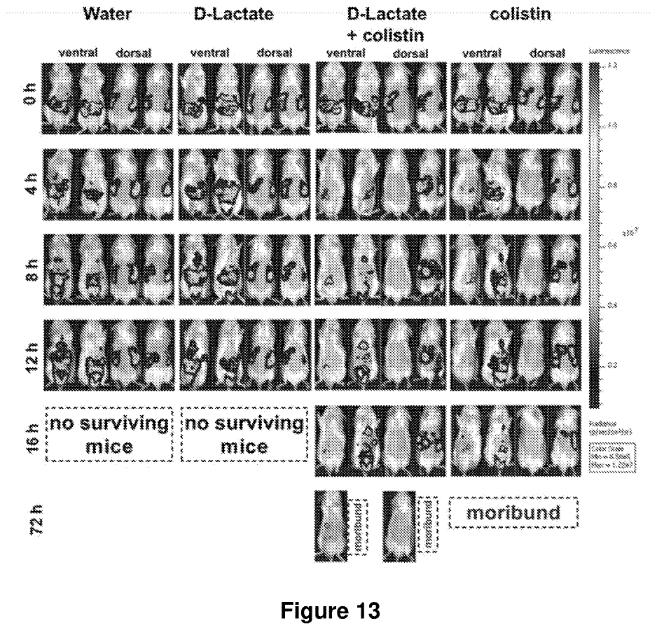

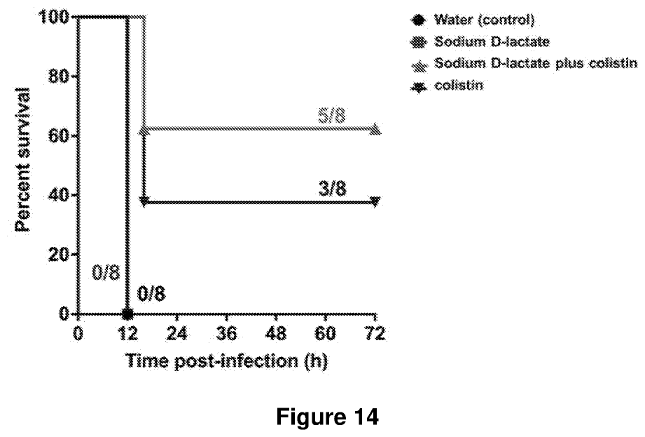

Treatment effectiveness was evaluated by: colony counts from blood samples taken at 4 h post-infection; imaging of infected mice for concentrations of the fluorescently-tagged col^{R} E. coli; and survival (FIGS. 12-14). Mice treated with a single dose of 1000 mg/kg Sodium D-Lactate plus four doses of 1 mg/kg colistin had significantly lower colony counts from blood samples than mice treated with four doses of 1 mg/kg colistin only. A single dose of 1000 mg/kg Sodium D-Lactate alone was not protective. Comparable effects were observed with both imaging of infected mice for concentrations of the fluorescently-tagged col^{R} E. coli and survival rates.

1. A composition for dysregulating the metabolism of planktonic bacteria, said composition comprising a 2-hydroxycarboxylic acid of the following formula:

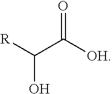

2. The composition of claim 1, wherein the 2-hydroxycarboxylic acid is a preferred enantiomer of a 2-hydroxycarboxylic acid with the following formula:

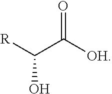

3. The composition of claim 2, further comprising an unpreferred enantiomer of a 2-hydroxycarboxylic acid with the following formula:

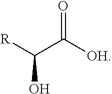

4. The composition of claim 3, wherein the % of unpreferred enantiomer of a 2-hydroxycarboxylic acid is less than 20% of the total amount of 2-hydroxycarboxylic acid.

5. The composition of claim 2, wherein the composition does not comprise an unpreferred enantiomer of a 2-hydroxycarboxylic acid.

6. The composition of claim 1, wherein the dysregulation is:
(i) suppression of metabolism in the presence of the 2-hydroxycarboxylic acid alone, and suppression of metabolism in the presence of the 2-hydroxycarboxylic acid and an antimicrobial compound;
(ii) suppression of metabolism in the presence of the 2-hydroxycarboxylic acid alone, but enhancement of metabolism in the presence of the 2-hydroxycarboxylic acid and an antimicrobial compound;
(iii) enhancement of metabolism in the presence of the 2-hydroxycarboxylic acid alone, and enhancement of metabolism in the presence of the 2-hydroxycarboxylic acid and an antimicrobial compound;
(iv) enhancement of metabolism in the presence of the 2-hydroxycarboxylic acid alone, but suppression of metabolism in the presence of the 2-hydroxycarboxylic acid and an antimicrobial compound;
(v) no effect on metabolism in the presence of the 2-hydroxycarboxylic acid alone, but suppression of metabolism in the presence of the 2-hydroxycarboxylic acid and an antimicrobial compound;
(vi) no effect on metabolism in the presence of the 2-hydroxycarboxylic acid alone, but enhancement of metabolism in the presence of the 2-hydroxycarboxylic acid and an antimicrobial compound.

7. The composition of claim 1, wherein the dysregulation results in the sensitisation of the bacteria to an antimicrobial compound.

8. The composition of claim 1, wherein the 2-hydroxycarboxylic acid is D-lactic acid or a pharmaceutically acceptable salt thereof.

9. The composition of claim 1, further comprising an antimicrobial compound.

10. A method to dysregulate the metabolism of planktonic bacteria comprising the step of: administering a composition comprising a 2-hydroxycarboxylic acid of any of claim 1 or 2 to the planktonic bacteria.

11. A composition comprising a 2-hydroxycarboxylic acid of any of claim 1 or 2 for use in treating or preventing a microbial infection in a subject, wherein the microbial infection is caused by planktonic bacteria and wherein the 2-hydroxycarboxylic acid dysregulates the metabolism of the planktonic bacteria.

12. The use of a 2-hydroxycarboxylic acid of any of claim 1 or 2 in the manufacture of a composition for dysregulating the metabolism of planktonic bacteria.

13. The use of composition comprising a 2-hydroxycarboxylic acid of any of claim 1 or 2 for dysregulating the metabolism of planktonic bacteria.

14. The use of a 2-hydroxycarboxylic acid of any of claim 1 or 2 in the manufacture of a composition for treating or preventing a microbial infection in a subject wherein the microbial infection is caused by planktonic bacteria and wherein the composition dysregulates the metabolism of the planktonic bacteria.

15. The use of composition comprising a 2-hydroxycarboxylic acid of any of claim 1 or 2 for treating or preventing a microbial infection in a subject wherein the microbial infection is caused by planktonic bacteria and wherein the 2-hydroxycarboxylic acid dysregulates the metabolism of the planktonic bacteria.

16. The use of composition comprising a 2-hydroxycarboxylic acid of any of claim 1 or 2 for dysregulating the metabolism of planktonic bacteria on a non-living surface or in a non-living substance.

17. A kit for dysregulating the metabolism of planktonic bacteria, said kit comprising:
a) a composition comprising a 2-hydroxycarboxylic acid of any of claim 1 or 2; and
b) instructions for use.

18. The method of claim 10, use of any of claims 11 to 16, or kit of claim 17, wherein the dysregulation is:
(i) suppression of metabolism in the presence of the 2-hydroxycarboxylic acid alone, and suppression of metabolism in the presence of the 2-hydroxycarboxylic acid and an antimicrobial compound;
(ii) suppression of metabolism in the presence of the 2-hydroxycarboxylic acid alone, but enhancement of metabolism in the presence of the 2-hydroxycarboxylic acid and an antimicrobial compound;
(iii) enhancement of metabolism in the presence of the 2-hydroxycarboxylic acid alone, and enhancement of metabolism in the presence of the 2-hydroxycarboxylic acid and an antimicrobial compound;
(iv) enhancement of metabolism in the presence of the 2-hydroxycarboxylic acid alone, but suppression of metabolism in the presence of the 2-hydroxycarboxylic acid and an antimicrobial compound;
(v) no effect on metabolism in the presence of the 2-hydroxycarboxylic acid alone, but suppression of metabolism in the presence of the 2-hydroxycarboxylic acid and an antimicrobial compound;
(vi) no effect on metabolism in the presence of the 2-hydroxycarboxylic acid alone, but enhancement of metabolism in the presence of the 2-hydroxycarboxylic acid and an antimicrobial compound.

19. The method of claim 10, use of any of claims 11 to 16, or kit of claim 17, wherein the dysregulation results in the sensitisation of the bacteria to an antimicrobial compound.

20. The method of claim 10, use of any of claims 11 to 16, or kit of claim 17, wherein the 2-hydroxycarboxylic acid is D-lactic acid or a pharmaceutically acceptable salt thereof.

21. The method of claim 10, use of any of claims 11 to 16, or kit of claim 17, wherein the composition further comprises an antimicrobial compound.
